(19) United States
(12) Patent Application Publication
(10) Pub. No.: US 2026/0207096 A1
(43) Pub. Date: Jul. 23, 2026

(54) ANALYTE SENSORS AND SENSING METHODS FOR DETECTING INHIBITORS OF DIAPHORASE

(71) Applicant: Abbott Diabetes Care Inc., Alameda, CA (US)

(72) Inventors: Benjamin J. FELDMAN, Berkeley, CA (US); Tianmei OUYANG, Fremont, CA (US); Zenghe LIU, Alameda, CA (US); Stephen OJA, Alameda, CA (US); Ahmed Hisham WALI, Alameda, CA (US); Cade Brylee FOX, Burlingame, CA (US)

(21) Appl. No.: 19/440,210

(22) Filed: Jan. 5, 2026

Related U.S. Application Data
(63) Continuation of application No. 17/314,146, filed on May 7, 2021, now abandoned.
(60) Provisional application No. 63/031,809, filed on May 29, 2020.

(51) Int. Cl. A61B 5/1486; A61B 5/145

(52) U.S. Cl. CPC A61B 5/14865; A61B 5/14532; A61B 2560/063

(57) ABSTRACT

Analyte sensors featuring an enzyme system comprising diaphorase and a NAD-dependent dehydrogenase may be utilized to detect inhibitors of diaphorase, provided that the transfer of electrons to a working electrode is rate-limiting with respect to the diaphorase. Such analyte sensors may comprise a sensor tail comprising at least a first working electrode, a first active area disposed upon a surface of the first working electrode, and an analyte-permeable membrane overcoating at least the first active area. The enzyme system comprises NAD, reduced NAD, or any combination thereof; a NAD-dependent dehydrogenase, such as NAD-dependent glucose dehydrogenase; and diaphorase. Inhibitors of diaphorase that may be detected include, for example, warfarin, dicoumarol, and similar compounds. A second active area may be present to facilitate detection of an analyte differing from the inhibitor of diaphorase.

### CROSS-REFERENCE TO RELATED APPLICATIONS

Not applicable.

### BACKGROUND

Detection of various analytes within an individual can sometimes be vital for monitoring the condition of their health and well-being. Some analytes are biomolecules produced internally, and their concentration may vary due to an underlying physiological condition or exposure to particular environmental factors. Drug or drug metabolite concentrations may similarly be analyzed as a measure of an individual's health and to aid medical personnel in making dosing and treatment decisions. Deviation from normal analyte levels can often be indicative of a worsening metabolic condition, illness, exposure to particular environmental conditions, or an ineffective treatment regimen. While a particular pathological source may dysregulate a single analyte in isolation, it is commonly the case that multiple analytes are concurrently dysregulated, either due to the same pathological source or a comorbid (related) condition. When multiple analytes are dysregulated, the extent of dysregulation may vary for each analyte. To achieve a complete evaluation of an individual's health, each analyte may need to be monitored.

Coumarin-based drugs, such as warfarin and dicoumarol, are commonly used anticoagulant drugs in patients having cardiovascular disease. Their mechanism of action involves competitive inhibition of vitamin K epoxide reductase, which depletes vitamin K in the blood and reduces blood clotting as a result. Despite their utility, it can be very difficult to maintain a therapeutically effective amount of coumarin-based drugs in vivo. Patients taking coumarin-based drugs must carefully regulate their diet to avoid foods rich in vitamin K, such as leafy green vegetables, to avoid reactivating the blood coagulation cycle and displacing the enzyme-bound coumarins. Further, patients may respond differently to coumarin-based drugs and/or metabolize coumarin-based drugs at widely different rates. Dangerous bleeding events may result if blood plasma levels of the coumarin-based drug become too high as a consequence of dosing that is too high or too frequent. Likewise, it is easy to fall below the therapeutically effective window for inhibiting blood coagulation as well. As still another difficulty, coumarin-based drugs, such as warfarin, can intensify the effect of some diabetes drugs and lead to extremely low blood sugar levels. Accordingly, medical personnel prescribing coumarin-based drugs usually have to carefully titrate up to a therapeutically effective dose for a particular patient and monitor for ongoing adverse side effects thereafter, particularly in diabetic patients.

Periodic, ex vivo analyte monitoring using a withdrawn bodily fluid can often be sufficient for monitoring the health of many individuals. Indeed, multiple blood draws may be needed when titrating coumarin-based drugs up to a therapeutically effective dose, within ongoing maintenance monitoring taking place thereafter. Since the dosing of coumarin-based drugs may frequently change, a significant number of blood draws for a patient may be needed over time. Not only can the multiple blood draws be painful, but they are frequently performed in a physician's office at set collection times, which may be inconvenient for a patient's work or personal schedule. In addition, the periodic nature of the blood draws may provide medical personnel with only a limited view of the in vivo profile of coumarin-based drugs and other analytes.

In vivo analyte sensors, particularly those employing enzyme-based detection to provide detection specificity, address some of the foregoing difficulties for certain analytes and are experiencing ever-increasing use. Indeed, in vivo analyte sensors utilizing a glucose-responsive enzyme for monitoring blood glucose levels are now in common use among diabetic individuals. Other types of analytes may be monitored using other enzymes or enzyme systems comprising multiple enzymes acting in concert. At present, however, there are relatively few in vivo analyte sensors featuring enzyme-based detection that can satisfactorily analyze for drugs or drug metabolites, such as coumarin-based drugs.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 shows a diagram of an illustrative sensing system that may incorporate an analyte sensor of the present disclosure.

FIGS. 2A-2C show enzyme systems configured for detecting ketones.

FIG. 3A shows how the enzyme system of FIG. 2A may be modified to detect glucose. FIG. 3B shows how the enzyme system of FIG. 3A may be further modified to detect an inhibitor of diaphorase. Thus, FIG. 3B shows an enzyme system configured for detecting an inhibitor of diaphorase.

FIGS. 4A-4C show cross-sectional diagrams of illustrative analyte sensors having an active area suitable for detecting an inhibitor of diaphorase.

FIGS. 5A-5C show cross-sectional diagrams of illustrative analyte sensors having a single working electrode and active areas suitable for detecting an inhibitor of diaphorase and another analyte.

FIG. 6 shows a cross-sectional diagram of an illustrative analyte sensor having two working electrodes and active areas suitable for detecting an inhibitor of diaphorase and another analyte.

FIGS. 7A-7C show perspective views of illustrative analyte sensors featuring electrodes that are disposed concentrically with respect to one another and containing active areas suitable for detecting an inhibitor of diaphorase and another analyte.

FIGS. 8A and 8B show enzyme systems configured for detecting glucose.

FIG. 9 shows an enzyme system configured for detecting creatinine.

FIG. 10 is a graph showing the results of titrating NADH and dicoumarol (DCM) into PBS solutions exposed to the analyte sensors of Example 1 (Sensors 1-4).

FIGS. 11A and 11B are graphs showing the results of titrating NADH and dicoumarol (DCM) into PBS solutions exposed to analyte sensors having a variable amount of electron transfer agent in an active area thereon.

FIG. 12 is a graph of sensor response as a function of NADH concentration for analyte sensors having a variable amount of electron transfer agent in an active area thereon.

FIG. 13 is a graph of normalized sensor response as a function of dicoumarol concentration for analyte sensors having a variable amount of electron transfer agent in an active area thereon.

FIGS. 14A and 14B are graphs showing the results of titrating glucose and dicoumarol (DCM) into PBS solutions exposed to the analyte sensors of Example 2 (Sensors 5-8).

### DETAILED DESCRIPTION

The present disclosure generally describes analyte sensors employing multiple enzymes for detection of one or more analytes and, more specifically, analyte sensors employing multiple enzymes acting in concert for detecting inhibitors of diaphorase, such as coumarin-based drugs, and corresponding methods for use thereof. Further analytes may be detected contemporaneously using a separate enzyme or enzyme system located upon the same analyte sensor.

As discussed above, analyte sensors employing enzyme-based detection are commonly used for assaying a single analyte, such as glucose or a related analyte, due to the frequent specificity of enzymes for a particular substrate or class of substrate. Analyte sensors employing both single enzymes and enzyme systems comprising multiple enzymes acting in concert may be used for this purpose. As used herein, the term “in concert” refers to a coupled enzymatic reaction, in which the product of a first enzymatic reaction becomes the substrate for a second enzymatic reaction, and the second enzymatic reaction or a subsequent enzymatic reaction serves as the basis for measuring the concentration of an analyte. In order to promote detection, the analyte may react during or otherwise impact at least one of the enzymatic reactions in the enzyme system. Using an in vivo analyte sensor featuring an enzyme or enzyme system to promote detection may be particularly advantageous to avoid the frequent withdrawal of bodily fluid that otherwise may be required for analyte monitoring to take place. Monitoring of drugs and drug metabolites using an in vivo analyte sensor may be especially problematic due to the rarity of identifying a suitable enzyme system for promoting specific detection of a particular drug or drug metabolite.

Coumarin-based drugs, such as warfarin and dicoumarol, are one class of drugs that would be highly desirable to monitor in vivo due to the difficulty of titrating and maintaining these drugs at a therapeutically effective level. At present, an effective manner for detecting and quantifying coumarin-based drugs or their metabolites in vivo is not believed to exist, particularly using an enzyme or enzyme system to facilitate detection. Vitamin K epoxide reductase, the target enzyme of some coumarin-based drugs, has not yet been exploited for a viable enzyme-based detection scheme for coumarin-based drugs.

Coumarin-based drugs, as well as several other types of compounds, are also very efficient inhibitors of the enzyme diaphorase. The present disclosure demonstrates that analyte sensors featuring an enzyme system comprising diaphorase may be configured to detect coumarin-based drugs effectively, as well as other inhibitors of the diaphorase enzyme. Enzyme systems may be electrically coupled to a working electrode to facilitate electrochemical analyte detection. Enzyme systems configured to detect coumarin-based drugs and similar inhibitors feature diaphorase and at least one additional enzyme acting in concert to generate an electrochemical signal at a working electrode. To facilitate detection of coumarin-based drugs and other inhibitors of diaphorase, the enzyme systems are made rate-limiting with respect to the diaphorase, such that the electrochemical signal (e.g., current) received at the working electrode may be correlated to the amount of coumarin-based drug or other diaphorase inhibitor that is present. Suitable enzyme systems comprising diaphorase and that are rate-limiting with respect to the diaphorase are described in further detail hereinbelow. Advantageously, such enzyme systems may take advantage of the high native concentration of glucose or other species in biological fluids to initiate an enzymatic cascade, eventually resulting in electron transfer to a working electrode, as also explained hereinbelow. As such, no additional reagents are needed to promote detection other than those housed within the analyte sensor itself.

In addition to detecting coumarin-based drugs and other inhibitors of diaphorase, the analyte sensors disclosed herein may also be further configured to detect one or more additional analytes as well. Illustrative examples of other analytes that may be analyzed using further detection chemistry housed within the same analyte sensor include, for example, glucose, ketones, creatinine, lactate, A1c, pH and the like. As mentioned above, in vivo analyte sensors featuring enzyme-based detection of glucose are now in wide use among diabetic individuals. Detection systems for the other analytes, one or more of which may be concurrently dysregulated in diabetic individuals, are also known. Detection systems for glucose or any one or more of the other foregoing analytes may also be incorporated within the analyte sensors disclosed herein in combination with the enzyme system configured for detecting inhibitors of diaphorase. Further details of how additional sensing chemistry may be incorporated within the analyte sensors of the present disclosure is provided hereinbelow.

The ability to monitor coumarin-based drugs in vivo represents a significant and advantageous clinical advance offered by the present disclosure. In addition, it may be further advantageous to monitor glucose levels in vivo in combination with analyzing for coumarin-based drugs due to the propensity of coumarin-based drugs to intensify the effect of diabetes drugs, which may lead to additional dosing dysregulation of the coumarin-based drug. Concurrently monitoring the concentration of glucose and coumarin-based drugs using an analyte sensor configured for detecting both analytes may provide a wealth of information to medical personnel and potentially afford improved patient outcomes. It may likewise be desirable to monitor other commonly dysregulated analytes in conjunction with monitoring the concentration of coumarin-based drugs and other inhibitors of diaphorase.

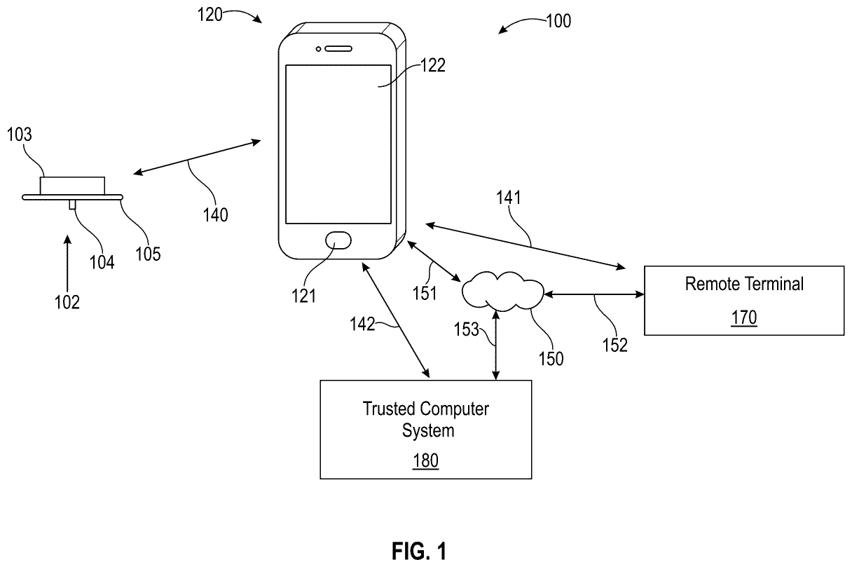

Before describing the analyte sensors of the present disclosure in further detail, a brief overview of suitable in vivo analyte sensor configurations and sensor systems employing the analyte sensors will be provided first so that the embodiments of the present disclosure may be better understood. FIG. 1 shows a diagram of an illustrative sensing system that may incorporate an analyte sensor of the present disclosure, specifically an analyte sensor comprising an active area responsive to an inhibitor of diaphorase. As shown, sensing system 100 includes sensor control device 102 and reader device 120 that are configured to communicate with one another over local communication path or link 140, which may be wired or wireless, uni- or bi-directional, and encrypted or non-encrypted. Reader device 120 may constitute an output medium for viewing analyte concentrations and alerts or notifications determined by sensor 104 or a processor associated therewith, as well as allowing for one or more user inputs, according to some embodiments. Reader device 120 may be a multi-purpose smartphone or a dedicated electronic reader instrument. While only one reader device 120 is shown, multiple reader devices 120 may be present in certain instances. Reader device 120 may also be in communication with remote terminal 170 and/or trusted computer system 180 via communication path(s)/link(s) 141 and/or 142, respectively, which also may be wired or wireless, uni- or bi-directional, and encrypted or non-encrypted. Reader device 120 may also or alternately be in communication with network 150 (e.g., a mobile telephone network, the internet, or a cloud server) via communication path/link 151. Network 150 may be further communicatively coupled to remote terminal 170 via communication path/link 152 and/or trusted computer system 180 via communication path/link 153. Alternately, sensor 104 may communicate directly with remote terminal 170 and/or trusted computer system 180 without an intervening reader device 120 being present. For example, sensor 104 may communicate with remote terminal 170 and/or trusted computer system 180 through a direct communication link to network 150, according to some embodiments, as described in U.S. Patent Application Publication 2011/0213225 and incorporated herein by reference in its entirety. Any suitable electronic communication protocol may be used for each of the communication paths or links, such as near field communication (NFC), radio frequency identification (RFID), BLUETOOTH® or BLUETOOTH® Low Energy protocols, WiFi, or the like. Remote terminal 170 and/or trusted computer system 180 may be accessible, according to some embodiments, by individuals other than a primary user who have an interest in the user's analyte levels. Reader device 120 may comprise display 122 and optional input component 121. Display 122 may comprise a touch-screen interface, according to some embodiments.

Sensor control device 102 includes sensor housing 103, which may house circuitry and a power source for operating sensor 104. Optionally, the power source and/or active circuitry may be omitted. A processor (not shown) may be communicatively coupled to sensor 104, with the processor being physically located within sensor housing 103 or reader device 120. Sensor 104 protrudes from the underside of sensor housing 103 and extends through adhesive layer 105, which is adapted for adhering sensor housing 103 to a tissue surface, such as skin, according to some embodiments.

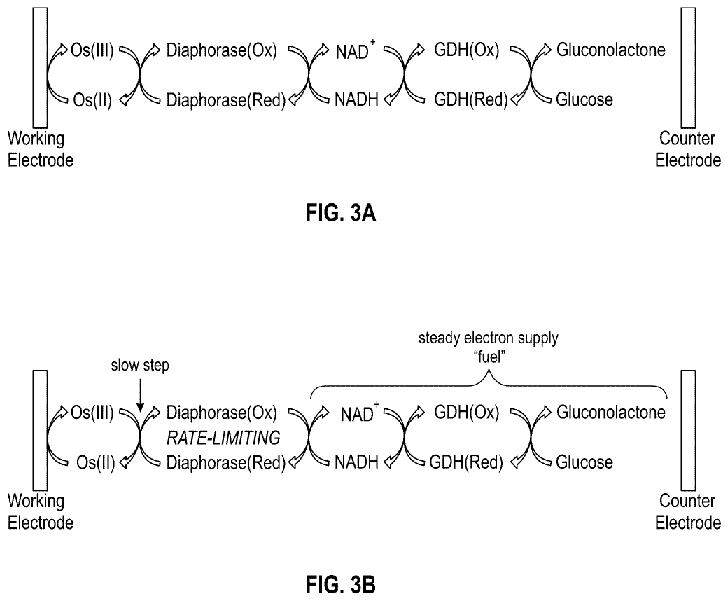

Sensor 104 is adapted to be at least partially inserted into a tissue of interest, such as within the dermal or subcutaneous layer of the skin. Sensor 104 may comprise a sensor tail of sufficient length for insertion to a desired depth in a given tissue. The sensor tail may comprise at least one working electrode and an active area comprising an enzyme system responsive to an inhibitor of diaphorase to facilitate detection of coumarin-based drugs and other inhibitors of diaphorase. Additional active areas to facilitate detection of one or more additional analytes may also be present, as specified in further detail herein. A counter electrode may be present in combination with the at least one working electrode, optionally in further combination with a reference electrode. Particular electrode configurations upon the sensor tail are described in more detail below in reference to FIGS. 3A-7C.

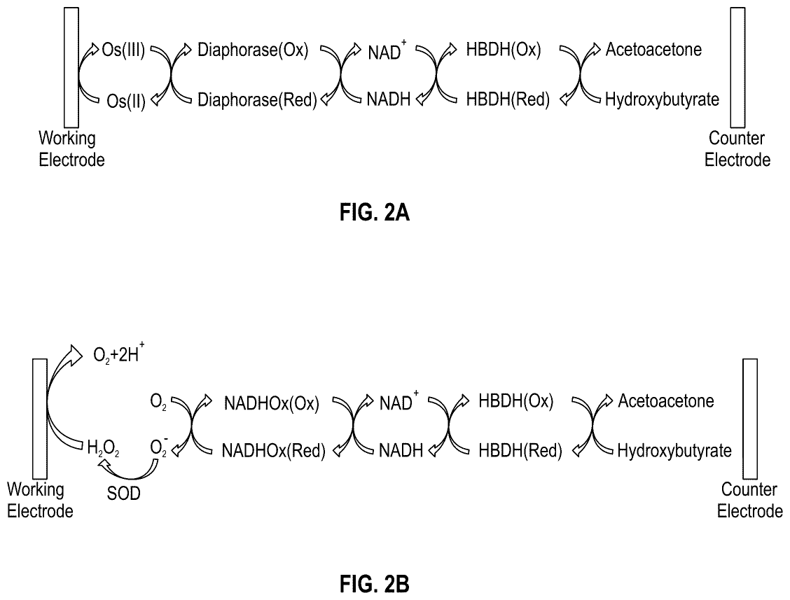

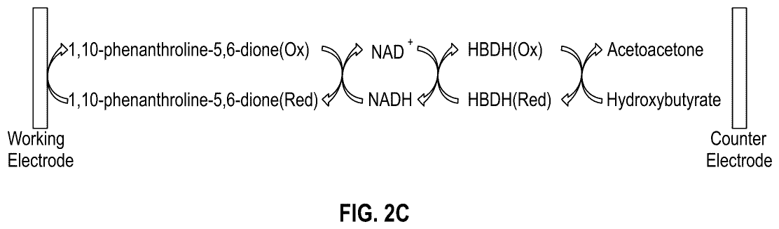

Active areas responsive to additional analytes may likewise feature a suitable enzyme or enzyme system for promoting detection of the additional analytes. If the active area responsive to another analyte is a glucose-responsive active area, for example, the glucose-responsive active area may comprise a glucose-responsive enzyme. Active areas responsive to other analytes may include those responsive to, for example, ketones, lactate, creatinine, pH or the like, which may feature separate enzymes or enzyme systems suitable for assaying these analytes. Suitable enzyme systems for detecting these analytes are described further below, particularly in reference to FIGS. 2A-2C, 8A, 8B and 9. One or more enzymes in the active area(s) may be covalently bonded to a polymer comprising the active area(s), according to various embodiments. The inhibitor of diaphorase and any additional analytes may be monitored in any biological fluid of interest such as dermal fluid, interstitial fluid, plasma, blood, lymph, synovial fluid, cerebrospinal fluid, saliva, bronchoalveolar lavage, amniotic fluid, or the like. In particular embodiments, analyte sensors of the present disclosure may be adapted for assaying dermal fluid or interstitial fluid to determine concentrations of the inhibitor of diaphorase and/or additional analytes in vivo.

One or more mass transport limiting membranes may overcoat the active area responsive to an inhibitor of diaphorase and an active area responsive to another analyte, if present. Analyte sensors oftentimes employ a membrane overcoating the active area(s) to limit mass transport and/or to improve biocompatibility. Mass transport limiting membranes may also be referred to as analyte-permeable membranes herein. Limiting analyte access to the active area(s) with a mass transport limiting membrane can aid in avoiding sensor overload (saturation), thereby improving detection performance and accuracy. When assaying multiple analytes using a single analyte sensor, different permeability values may be exhibited by the various analytes across a given mass transport limiting membrane, potentially resulting in different sensitivities for each analyte. Advantageously, sensor architectures are available for incorporating different mass transport limiting membranes upon each active area, when needed, to facilitate detection of multiple analytes. If a single mass transport limiting membrane provides satisfactory permeability for both analytes, a simpler sensor architecture may be used.

Referring again to FIG. 1, sensor 104 may automatically forward data to reader device 120. For example, analyte concentration data (i.e., coumarin-based drug concentrations and/or glucose, ketones, lactate, or creatinine concentrations, or pH values) may be communicated automatically and periodically, such as at a certain frequency as data is obtained or after a certain time period has passed, with the data being stored in a memory until transmittal (e.g., every minute, five minutes, or other predetermined time period). In other embodiments, sensor 104 may communicate with reader device 120 in a non-automatic manner and not according to a set schedule. For example, data may be communicated from sensor 104 using RFID technology when the sensor electronics are brought into communication range of reader device 120. Until communicated to reader device 120, data may remain stored in a memory of sensor 104. Thus, a user does not have to maintain close proximity to reader device 120 at all times, and can instead upload data at a convenient time. In yet other embodiments, a combination of automatic and non-automatic data transfer may be implemented. For example, data transfer may continue on an automatic basis until reader device 120 is no longer in communication range of sensor 104.

An introducer may be present transiently to promote introduction of sensor 104 into a tissue. In illustrative embodiments, the introducer may comprise a needle or similar sharp. It is to be recognized that other types of introducers, such as sheaths or blades, may be present in alternative embodiments. More specifically, the needle or other introducer may transiently reside in proximity to sensor 104 prior to tissue insertion and then be withdrawn afterward. While present, the needle or other introducer may facilitate insertion of sensor 104 into a tissue by opening an access pathway for sensor 104 to follow. For example, the needle may facilitate penetration of the epidermis as an access pathway to the dermis to allow implantation of sensor 104 to take place, according to one or more embodiments. After opening the access pathway, the needle or other introducer may be withdrawn so that it does not represent a sharps hazard. In illustrative embodiments, suitable needles may be solid or hollow, beveled or non-beveled, and/or circular or non-circular in cross-section. In more particular embodiments, suitable needles may be comparable in cross-sectional diameter and/or tip design to an acupuncture needle, which may have a cross-sectional diameter of about 250 microns. It is to be recognized, however, that suitable needles may have a larger or smaller cross-sectional diameter if needed for particular applications.

In some embodiments, a tip of the needle (while present) may be angled over the terminus of sensor 104, such that the needle penetrates a tissue first and opens an access pathway for sensor 104. In other illustrative embodiments, sensor 104 may reside within a lumen or groove of the needle, with the needle similarly opening an access pathway for sensor 104. In either case, the needle may be subsequently withdrawn after facilitating sensor insertion.

FIG. 2A shows an enzyme system configured for detecting ketones. Additional enzyme systems suitable for detecting ketones are shown in FIGS. 2B and 2C, which are described further below. In the enzyme system shown in FIG. 2A, β-hydroxybutyrate serves as a surrogate for ketones formed in vivo, which undergoes a reaction with an enzyme system comprising β-hydroxybutyrate dehydrogenase (HBDH) and diaphorase to facilitate ketones detection within a ketones-responsive active area disposed upon the surface of at least one working electrode, as described further herein. Within the ketones-responsive active area, β-hydroxybutyrate dehydrogenase may convert β-hydroxybutyrate and oxidized nicotinamide adenine dinucleotide (NAD⁺) into acetoacetate and reduced nicotinamide adenine dinucleotide (NADH), respectively. It is to be understood that the term “nicotinamide adenine dinucleotide (NAD)” includes a phosphate-bound form of the foregoing enzyme cofactors. That is, use of the term “NAD” herein refers to both NAD⁺ phosphate and NADH phosphate, specifically a diphosphate linking the two nucleotides, one containing an adenine nucleobase and the other containing a nicotinamide nucleobase. The NAD⁺ and NADH enzyme cofactors aid in promoting the concerted enzymatic reactions disclosed herein. Once formed, the NADH may undergo oxidation under diaphorase mediation, with the electrons transferred during this process providing the basis for ketone detection at the working electrode. Thus, there is a 1:1 molar correspondence between the amount of electrons transferred to the working electrode and the amount of β-hydroxybutyrate converted, thereby providing the basis for ketones detection and quantification based upon the measured amount of current at the working electrode. Transfer of the electrons to the working electrode may take place under further mediation of an electron transfer agent, such as an osmium (Os) compound or similar transition metal complex, as described in additional detail below. Albumin may further be present as a stabilizer within the active area. The β-hydroxybutyrate dehydrogenase and the diaphorase may be covalently bonded to a polymer comprising the ketones-responsive active area. The NAD⁺ may or may not be covalently bonded to the polymer, but if the NAD⁺ is not covalently bonded, it may be physically retained within the ketones-responsive active area, such as with a mass transport limiting membrane overcoating the ketones-responsive active area, wherein the mass transport limiting membrane is also permeable to ketones.

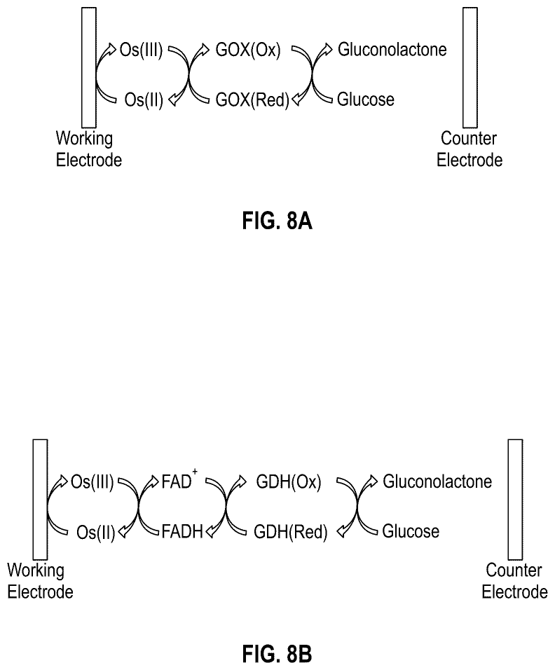

The present disclosure demonstrates how the enzyme system shown in FIG. 2A can be modified to become responsive to other analytes. FIGS. 3A and 3B show how the enzyme system of FIG. 2A may be sequentially modified to become responsive to glucose and an inhibitor of diaphorase, respectively. As shown in FIG. 3A, by replacing β-hydroxybutyrate dehydrogenase with NAD-dependent glucose dehydrogenase in the active area, the analyte sensor may become responsive to glucose, in which case gluconolactone is formed as a product of glucose oxidation. Diaphorase may facilitate the transfer of electrons between the NAD and the electron transfer agent. Even simpler enzyme-based detection schemes for glucose are shown in FIGS. 8A and 8B below, in which glucose oxidase or FAD-dependent glucose dehydrogenase may transfer electrons to an electron transfer agent without an additional enzyme being present.

The enzyme system depicted in FIG. 3A may be linearly responsive to glucose, provided that there is sufficient turnover of the downstream enzyme and cofactor (diaphorase and NAD⁺/NADH, respectively) to facilitate transfer of all the electrons generated during glucose oxidation to the working electrode. In the present disclosure, the enzyme system of FIG. 3A may be further modified to make the diaphorase rate-limiting with respect to electron transfer to the working electrode. By making the diaphorase rate-limiting, the diaphorase serves as a “valve” for controlling the flow of electrons to the working electrode. With the diaphorase being rate-limiting, a constant signal results, regardless of the glucose concentration upstream of the diaphorase. However, with the diaphorase being rate-limiting, an inhibitor of diaphorase, such as coumarin-based drugs and other inhibitors of diaphorase, may alter the flow of electrons to the working electrode and serve as a basis for detection of the inhibitor. More specifically, a decrease in the flow of electrons to the working electrode may be correlated to the amount of inhibitor present. Moreover, since glucose is ubiquitously present in biological fluids, the glucose may serve as a “fuel” for providing a steady flow of electrons to the rate-limiting diaphorase enzyme. Accordingly, FIG. 3B shows an enzyme system configured for detecting an inhibitor of diaphorase, which bears these considerations in mind. Other NAD-dependent dehydrogenases may be used as an alternative to NAD-dependent glucose dehydrogenase in FIG. 3B, provided that a ready supply of a substrate thereof exists in a biological fluid being analyzed.

Accordingly, analyte sensors of the present disclosure may comprise a sensor tail comprising at least a first working electrode, a first active area disposed upon a surface of the first working electrode, and an analyte-permeable membrane overcoating at least the first active area. The enzyme system comprises nicotinamide adenine dinucleotide (NAD), reduced nicotinamide adenine dinucleotide (NADH), or any combination thereof; a NAD-dependent dehydrogenase; and diaphorase; wherein transfer of electrons from the first active area to the first working electrode is rate-limiting with respect to the diaphorase, such that the first active area is responsive to an inhibitor of diaphorase, as explained above. Optionally, the analyte-permeable membrane overcoating the first active area may be omitted if the NAD or NADH can be adequately retained within the first active area, such as through physical entrainment or covalent bonding to a polymer comprising the first active area.

In a particular example, the NAD-dependent dehydrogenase may be NAD-dependent glucose dehydrogenase, wherein glucose present in a fluid undergoing analysis may provide a source of electrons to the working electrode. Other NAD-dependent dehydrogenases may be utilized similarly, provided that a ready supply of a substrate thereof exists in a fluid undergoing analysis, particularly a biological fluid.

To make the analyte sensor responsive to an inhibitor of diaphorase, the first active area may comprise the diaphorase in a rate-limiting amount with respect to transferring electrons to the first working electrode, the diaphorase may be modified to become rate-limiting with respect to transferring electrons to the first working electrode, or any combination thereof. Modification of a wild-type diaphorase into a modified diaphorase having reduced activity may be conducted, for example.

As mentioned above, inhibitors of diaphorase that may be monitored using the analyte sensors disclosed herein include warfarin and dicoumarol. Other diaphorase inhibitors may be assayed using the analyte sensors disclosed herein include, for example, N-methylmaleimide, diphenyleneiodonium, 5,6-dimethylxanthenone-4-acetic acid, flavone-8-acetic acid, dimethylbenzylalkammonium chloride, 7,8-dihydroxyflavone, chrysin, and any combination thereof.

The analyte sensors disclosed herein feature at least an active area responsive to an inhibitor of diaphorase upon a working electrode, in combination with at least one additional electrode, which may be a counter electrode, a reference electrode, and/or a counter/reference electrode. Additional working electrodes may be present in some cases. Analyte sensors featuring an active area responsive to an inhibitor of diaphorase in combination with an active area responsive to another analyte, such as glucose, are also contemplated by the present disclosure and are discussed further herein. Illustrative analyte sensor configurations adapted to assay one or multiple analytes are discussed further hereinbelow.

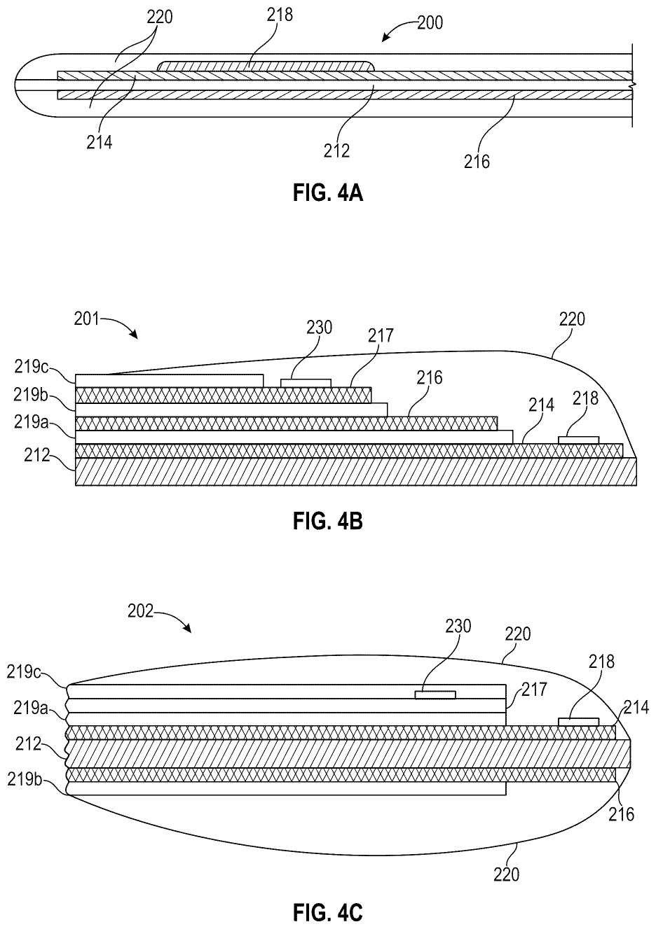

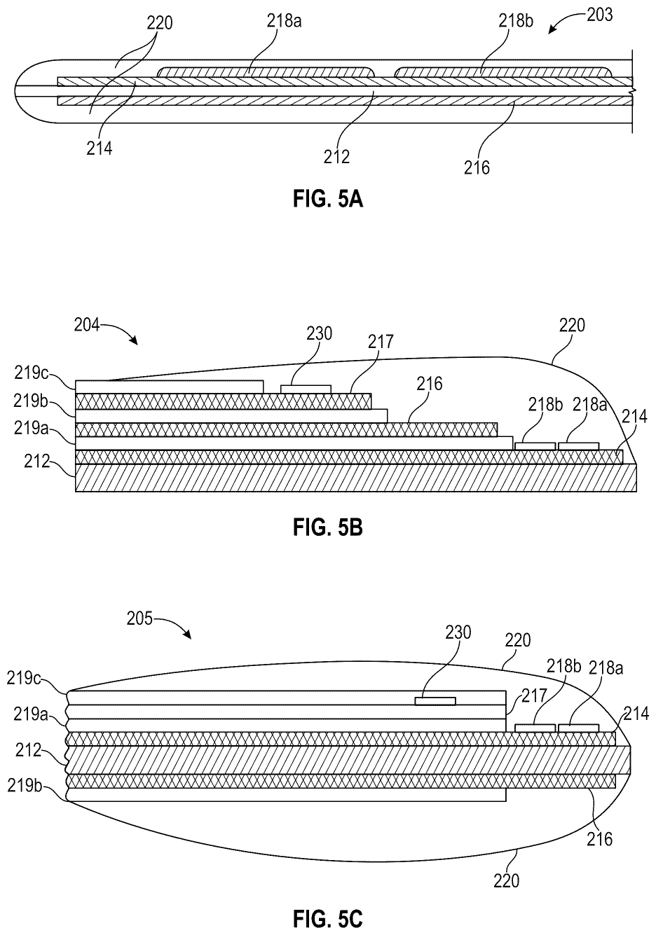

Sensor configurations featuring an active area responsive to an inhibitor of diaphorase but not an active area responsive to another analyte may employ two-electrode or three-electrode detection motifs, as described further herein in reference to FIGS. 4A-4C. Sensor configurations featuring both an active area responsive to an inhibitor of diaphorase and an active area responsive to another analyte, either upon separate working electrodes or upon the same working electrode, are described separately thereafter in reference to FIGS. 5A-7C. Sensor configurations having multiple working electrodes may be particularly advantageous for analyte sensors containing active areas configured to monitor two or more different analytes within the same sensor tail, since the signal contribution from each active area may be determined more readily.

When a single working electrode is present in an analyte sensor, three-electrode sensor configurations may comprise a working electrode, a counter electrode, and a reference electrode. Related two-electrode sensor configurations may comprise a working electrode and a second electrode, in which the second electrode may function as both a counter electrode and a reference electrode (i.e., a counter/reference electrode). The various electrodes may be at least partially stacked (layered) upon one another and/or laterally spaced apart from one another upon the sensor tail. Suitable sensor configurations may be substantially flat in shape or substantially cylindrical in shape. In any of the sensor configurations disclosed herein, the various electrodes may be electrically isolated from one another by a dielectric material or similar insulator. Analyte sensors containing an active area responsive to an inhibitor of diaphorase and an active area responsive to another analyte, such as glucose, may feature the active areas laterally spaced apart upon the working electrode.

Analyte sensors featuring multiple working electrodes may similarly comprise at least one additional electrode. When one additional electrode is present, the one additional electrode may function as a counter/reference electrode for each of the multiple working electrodes. When two additional electrodes are present, one of the additional electrodes may function as a counter electrode for each of the multiple working electrodes and the other of the additional electrodes may function as a reference electrode for each of the multiple working electrodes.

FIG. 4A shows a diagram of an illustrative two-electrode analyte sensor configuration, which is compatible for use in the disclosure herein. As shown, analyte sensor 200 comprises substrate 212 disposed between working electrode 214 and counter/reference electrode 216. Alternately, working electrode 214 and counter/reference electrode 216 may be located upon the same side of substrate 212 with a dielectric material interposed in between (configuration not shown). Active area 218, which is responsive to an inhibitor of diaphorase, is disposed as at least one layer upon at least a portion of working electrode 214. Active area 218 may comprise multiple spots or a single spot configured for detection of an inhibitor of diaphorase, as discussed further herein.

Referring still to FIG. 4A, membrane 220 may overcoat at least active area 218 and may optionally overcoat some or all of working electrode 214 and/or counter/reference electrode 216, or the entirety of analyte sensor 200, according to some embodiments. One or both faces of analyte sensor 200 may be overcoated with membrane 220. Membrane 220 may comprise one or more polymeric membrane materials having capabilities of limiting analyte flux to active area 218 (i.e., membrane 220 is a mass transport limiting membrane having some permeability for an inhibitor of diaphorase). The composition and thickness of membrane 220 may vary to promote a desired diaphorase inhibitor flux to active area 218. In non-limiting examples, membrane 220 may be coated onto active area 218 by one or more of spray coating, dip coating, printing and/or similar deposition techniques. The membrane thickness may be selected such that the current produced at working electrode 214 remains correlatable to the amount of diaphorase inhibitor that is present. Analyte sensor 200 may be operable for assaying the inhibitor of diaphorase by any of coulometric, amperometric, voltammetric, or potentiometric electrochemical detection techniques.

FIGS. 4B and 4C show diagrams of illustrative three-electrode analyte sensor configurations, which are also compatible for use in the disclosure herein. Three-electrode analyte sensor configurations may be similar to that shown for analyte sensor 200 in FIG. 4A, except for the inclusion of additional electrode 217 in analyte sensors 201 and 202 (FIGS. 4B and 4C). With additional electrode 217, counter/reference electrode 216 may then function as either a counter electrode or a reference electrode, and additional electrode 217 fulfills the other electrode function not otherwise accounted for. Working electrode 214 continues to fulfill its original function. Additional electrode 217 may be disposed upon either working electrode 214 or electrode 216, with a separating layer of dielectric material in between. For example, as depicted in FIG. 4B, dielectric layers 219a, 219b and 219c separate electrodes 214, 216 and 217 from one another and provide electrical isolation. Alternately, at least one of electrodes 214, 216 and 217 may be located upon opposite faces of substrate 212, as shown in FIG. 4C. Thus, in some embodiments, electrode 214 (working electrode) and electrode 216 (counter electrode) may be located upon opposite faces of substrate 212, with electrode 217 (reference electrode) being located upon one of electrodes 214 or 216 and spaced apart therefrom with a dielectric material. Reference material layer 230 (e.g., Ag/AgCl) may be present upon electrode 217, with the location of reference material layer 230 not being limited to that depicted in FIGS. 4B and 4C. As with sensor 200 shown in FIG. 4A, active area 218 in analyte sensors 201 and 202 may comprise multiple spots or a single spot. Analyte sensors 201 and 202 may likewise be operable for assaying an inhibitor of diaphorase by any of coulometric, amperometric, voltammetric, or potentiometric electrochemical detection techniques.

Like analyte sensor 200, membrane 220 may also overcoat active area 218, as well as other sensor components, in analyte sensors 201 and 202, thereby serving as a mass transport limiting membrane. Additional electrode 217 may be overcoated with membrane 220 in some embodiments. Although FIGS. 4B and 4C have depicted all of electrodes 214, 216 and 217 as being overcoated with membrane 220, it is to be recognized that only working electrode 214 may be overcoated in some embodiments. Moreover, the thickness of membrane 220 at each of electrodes 214, 216 and 217 may be the same or different. In non-limiting examples, membrane 220 may be coated onto active area 218 by one or more of spray coating, dip coating, printing and/or similar deposition techniques. As in two-electrode analyte sensor configurations (FIG. 4A), one or both faces of analyte sensors 201 and 202 may be overcoated with membrane 220 in the sensor configurations of FIGS. 4B and 4C, or the entirety of analyte sensors 201 and 202 may be overcoated. Accordingly, the three-electrode sensor configurations shown in FIGS. 4B and 4C should be understood as being non-limiting of the embodiments disclosed herein, with alternative electrode and/or layer configurations remaining within the scope of the present disclosure.

Analyte sensors having both an active area responsive to an inhibitor of diaphorase and an active area responsive to another analyte, each being located upon a single working electrode or upon multiple working electrodes, are described in further detail in reference to FIGS. 5A-7C.

FIG. 5A shows an illustrative configuration for sensor 203 having a single working electrode with both an active area responsive to an inhibitor of diaphorase and an active area responsive to another analyte disposed thereon. FIG. 5A is similar to FIG. 4A, except for the presence of two active areas upon working electrode 214: active area 218a (responsive to an inhibitor of diaphorase) and active area 218b (responsive to another analyte), which are laterally spaced apart from one another upon the surface of working electrode 214. Active areas 218a and 218b may comprise multiple spots or a single spot configured for detection of each analyte. The composition of membrane 220 may vary or be compositionally the same at active areas 218a and 218b. For example, when membrane 220 varies compositionally at active areas 218a and 218b, a single membrane polymer may be present at one of the active areas (e.g., active area 218b) and a bilayer of the membrane polymer or an admixture of the membrane polymer may be present at the other of the active areas (e.g., active area 218a). One or more of spray coating, dip coating, printing and/or similar deposition techniques may be used to deposit a compositionally homogeneous or compositionally differing membrane 220 at active areas 218a and 218b. First active area 218a and second active area 218b may be configured to detect their corresponding analytes at working electrode potentials that differ from one another, as discussed further below.

FIGS. 5B and 5C show cross-sectional diagrams of illustrative three-electrode sensor configurations for sensors 204 and 205, respectively, each featuring a single working electrode having both active area 218a (responsive to an inhibitor of diaphorase) and active area 218b (responsive to another analyte) disposed thereon. FIGS. 5B and 5C are otherwise similar to FIGS. 4B and 4C, respectively, and may be better understood by reference thereto. As with FIG. 5A, the composition of membrane 220 may be compositionally the same or vary at active areas 218a and 218b.

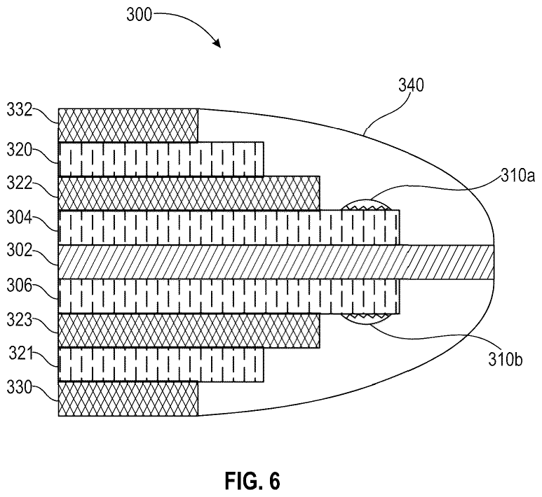

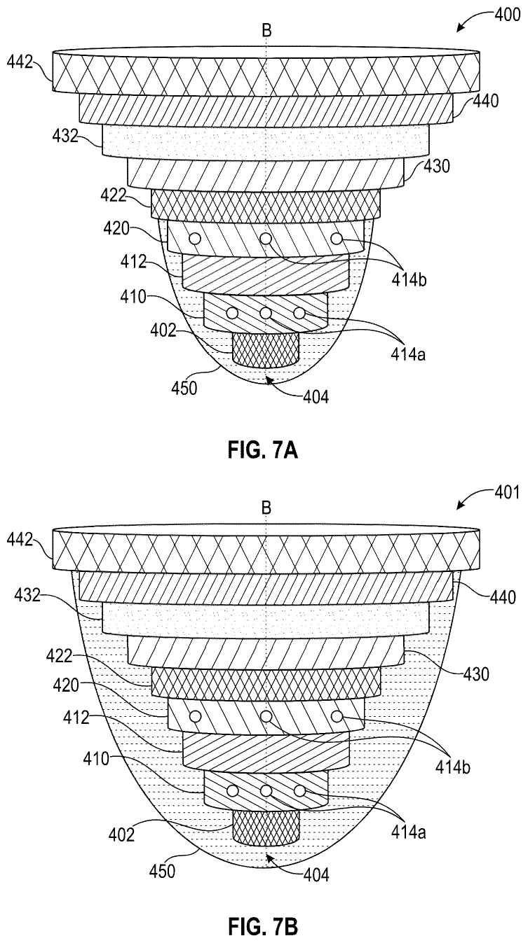

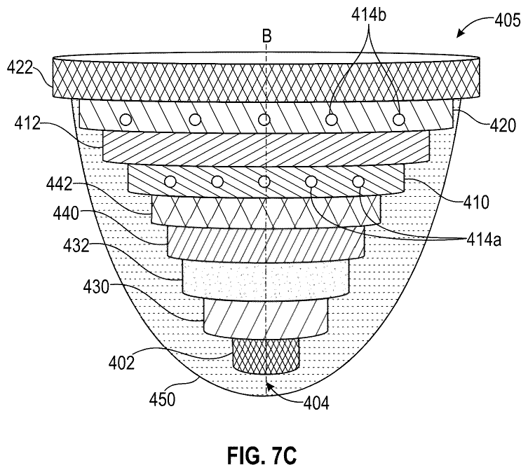

Illustrative sensor configurations having multiple working electrodes, specifically two working electrodes, are described in further detail in reference to FIGS. 6-7C. Although the following description is primarily directed to sensor configurations having two working electrodes, it is to be appreciated that more than two working electrodes may be incorporated through extension of the disclosure herein. Additional working electrodes may be used to impart additional sensing capabilities to the analyte sensors beyond just that of an inhibitor of diaphorase and one additional analyte. That is, analyte sensors containing more than two working electrodes may be suitable for detecting a commensurate number of additional analytes.

FIG. 6 shows a cross-sectional diagram of an illustrative analyte sensor configuration having two working electrodes, a reference electrode and a counter electrode, which may be compatible for use in the disclosure herein. As shown, analyte sensor 300 includes working electrodes 304 and 306 disposed upon opposite faces of substrate 302. Active area 310a (responsive to an inhibitor of diaphorase) is disposed upon the surface of working electrode 304, and active area 310b (responsive to another analyte) is disposed upon the surface of working electrode 306. Counter electrode 320 is electrically isolated from working electrode 304 by dielectric layer 322, and reference electrode 321 is electrically isolated from working electrode 306 by dielectric layer 323. Outer dielectric layers 330 and 332 are positioned upon reference electrode 321 and counter electrode 320, respectively. Membrane 340 may overcoat at least active areas 310a and 310b, according to various embodiments, with other components of analyte sensor 300 or the entirety of analyte sensor 300 optionally being overcoated with membrane 340 as well. Again, membrane 340 may be compositionally the same or vary compositionally at active areas 310a and 310b, if needed, in order to regulate the analyte flux at each location. Compositional variation may include admixtures of multiple membrane polymers or bilayers of multiple membrane polymers, for example.

Alternative sensor configurations having multiple working electrodes and differing from the configuration shown in FIG. 6 may feature a counter/reference electrode instead of separate counter and reference electrodes 320,321, and/or feature layer and/or membrane arrangements varying from those expressly depicted. For example, the positioning of counter electrode 320 and reference electrode 321 may be reversed from that depicted in FIG. 6. In addition, working electrodes 304 and 306 need not necessarily reside upon opposing faces of substrate 302 in the manner shown in FIG. 6.

Although suitable sensor configurations may feature electrodes that are substantially planar in character, it is to be appreciated that sensor configurations featuring non-planar electrodes may be advantageous and particularly suitable for use in the disclosure herein. In particular, substantially cylindrical electrodes that are disposed concentrically with respect to one another may facilitate deposition of a mass transport limiting membrane differing in composition at two different active areas, as described hereinbelow. FIGS. 7A-7C show perspective views of analyte sensors featuring two working electrodes that are disposed concentrically with respect to one another. It is to be appreciated that sensor configurations having a concentric electrode disposition but lacking a second working electrode are also possible in the present disclosure.

FIG. 7A shows a perspective view of an illustrative sensor configuration in which multiple electrodes are substantially cylindrical and are disposed concentrically with respect to one another about a central substrate. As shown, analyte sensor 400 includes central substrate 402 about which all electrodes and dielectric layers are disposed concentrically with respect to one another. In particular, working electrode 410 is disposed upon the surface of central substrate 402, and dielectric layer 412 is disposed upon a portion of working electrode 410 distal to sensor tip 404. Working electrode 420 is disposed upon dielectric layer 412, and dielectric layer 422 is disposed upon a portion of working electrode 420 distal to sensor tip 404. Counter electrode 430 is disposed upon dielectric layer 422, and dielectric layer 432 is disposed upon a portion of counter electrode 430 distal to sensor tip 404. Reference electrode 440 is disposed upon dielectric layer 432, and dielectric layer 442 is disposed upon a portion of reference electrode 440 distal to sensor tip 404. As such, exposed surfaces of working electrode 410, working electrode 420, counter electrode 430, and reference electrode 440 are spaced apart from one another along longitudinal axis B of analyte sensor 400. The surface areas of working electrode 410, working electrode 420, counter electrode 430, and reference electrode 440 progressively increase in size moving away from sensor tip 404.

Referring still to FIG. 7A, active areas 414a (responsive to an inhibitor of diaphorase) and active area 414b (responsive to another analyte) are disposed upon the exposed surfaces of working electrodes 410 and 420, respectively, thereby allowing contact with a fluid to take place for sensing of both analytes. Although active area 414a and 414b have been depicted as three discrete spots in FIG. 7A, it is to be appreciated that fewer or greater than three spots may be present in alternative sensor configurations. Moreover, the positioning of active area 414a and active area 414b may be reversed from that depicted in FIG. 7A.

In FIG. 7A, sensor 400 is partially coated with membrane 450 upon working electrodes 410 and 420 and active area 414a and 414b disposed thereon. FIG. 7B shows an alternative sensor configuration in which the substantial entirety of sensor 401 is overcoated with membrane 450. Membrane 450 may be the same or vary compositionally at active area 414a and 414b. Dip coating techniques may be particularly desirable for applying the membrane in a substantially cylindrical sensor configuration.

It is to be further appreciated that the positioning of the various electrodes in FIGS. 7A and 7B may differ from that expressly depicted. For example, the positions of counter electrode 430 and reference electrode 440 may be reversed from the depicted configurations in FIGS. 7A and 7B. Similarly, the positions of working electrodes 410 and 420 are not limited to those that are expressly depicted in FIGS. 7A and 7B. FIG. 7C shows an alternative sensor configuration to that shown in FIG. 7B, in which sensor 405 contains counter electrode 430 and reference electrode 440 that are located more proximal to sensor tip 404 and working electrodes 410 and 420 that are located more distal to sensor tip 404. Sensor configurations in which working electrodes 410 and 420 are located more distal to sensor tip 404 may be advantageous by providing a larger surface area for deposition of active area 414a and 414b (five discrete sensing spots illustratively shown for each in FIG. 7C), thereby facilitating an increased signal strength in some cases.

Although FIGS. 7A-7C have depicted sensor configurations that are each supported upon central substrate 402, it is to be appreciated that alternative sensor configurations may be electrode-supported instead and lack central substrate 402 (configuration not shown). In particular, the innermost concentric electrode may be utilized to support the other electrodes and dielectric layers. For example, counter electrode 430 may be the innermost concentric electrode and be employed for disposing thereon reference electrode 440, working electrodes 410 and 420, and dielectric layers 432, 442, 412, and 422. In view of the disclosure herein, it is again to be appreciated that other electrode and dielectric layer configurations may be employed in sensor configurations lacking central substrate 402.

Accordingly, analyte sensors of the present disclosure may further comprise an active area responsive to an analyte differing from the inhibitor of diaphorase, which is also disposed upon the sensor tail. Accordingly, analyte sensors of the present disclosure may be configured for analyzing for multiple analytes in particular embodiments. Other analytes that may be monitored in addition to the inhibitor of diaphorase include, for example, glucose, ketones, lactate, creatinine, pH, or any combination thereof. Suitable enzymes, enzyme systems or similar detection protocols for assaying these additional analytes in an analyte sensor are discussed further below.

In some embodiments, the analyte sensors may further comprise a glucose-responsive active area comprising a glucose-responsive enzyme disposed upon the sensor tail. Suitable glucose-responsive enzymes may include, for example, glucose oxidase or a glucose dehydrogenase (e.g., pyrroloquinoline quinone (PQQ) or a cofactor-dependent glucose dehydrogenase, such as flavine adenine dinucleotide (FAD)-dependent glucose dehydrogenase or nicotinamide adenine dinucleotide (NAD)-dependent glucose dehydrogenase). Glucose oxidase and glucose dehydrogenase are differentiated by their ability to utilize oxygen as an electron acceptor when oxidizing glucose; glucose oxidase may utilize oxygen as an electron acceptor, whereas glucose dehydrogenases transfer electrons to natural or artificial electron acceptors, such as an enzyme cofactor. Illustrative enzyme-based detection schemes for analyzing glucose are further shown in FIGS. 3A, 8A and 8B, which utilize glucose oxidase or glucose dehydrogenase to promote detection. Both glucose oxidase and glucose dehydrogenase may be covalently bonded to a polymer comprising the glucose-responsive active area and exchange electrons with an electron transfer agent (e.g., an osmium (Os) complex or similar transition metal complex), which may also be covalently bonded to the polymer. Suitable electron transfer agents are described in further detail below. Glucose oxidase may directly exchange electrons with the electron transfer agent (FIG. 8A), whereas glucose dehydrogenase may utilize a cofactor to promote electron exchange with the electron transfer agent (FIGS. 3A and 8B). FAD cofactor may directly exchange electrons with the electron transfer agent, as shown in FIG. 8B. NAD cofactor, in contrast, may utilize diaphorase to facilitate electron transfer from the cofactor to the electron transfer agent, as shown in FIG. 3A and described further above. Further details concerning glucose-responsive active areas incorporating glucose oxidase or glucose dehydrogenase, as well as glucose detection therewith, may be found in commonly owned U.S. Pat. No. 8,268,143, for example.

Concurrent detection of an inhibitor of diaphorase and glucose may be particularly desirable due to the propensity for dicoumarol and other coumarin-based drugs to impact the activity of certain diabetes drugs. As such, analyte sensors capable of analyzing for both a diaphorase inhibitor and glucose may facilitate treatment decisions and potentially improve patient outcomes. Considerations for detecting a second analyte, such as glucose, in combination with an inhibitor or diaphorase, are provide below.

In some embodiments, the analyte sensors may further comprise a ketones-responsive active area comprising an enzyme system that operates in concert to facilitate detection of ketones. Suitable enzyme systems for facilitating detection of ketones are described above in reference to FIG. 2A. Additional enzyme systems that may operate in concert to facilitate detection of ketones are shown in FIGS. 2B and 2C. In FIGS. 2B and 2C, there is again a 1:1 molar correspondence between the amount of electrons transferred to the working electrode and the amount of β-hydroxybutyrate converted, thereby providing the basis for ketones detection. Additional details concerning enzyme systems responsive to ketones may be found in commonly owned U.S. patent application Ser. No. 16/774,835 entitled “Analyte Sensors and Sensing Methods Featuring Dual Detection of Glucose and Ketones,” filed on Jan. 28, 2020, and published as U.S. Patent Application Publication 2020/0237275, the entirety of which is incorporated herein by reference.

As shown in FIG. 2B, β-hydroxybutyrate dehydrogenase (HBDH) may again convert β-hydroxybutyrate and NAD⁺ into acetoacetate and NADH, respectively. Instead of electron transfer to the working electrode being completed by diaphorase (see FIG. 2A) and a transition metal electron transfer agent, the reduced form of NADH oxidase (NADHOx (Red)) undergoes a reaction to form the corresponding oxidized form (NADHOX (Ox)). NADHOx (Red) may then reform through a reaction with molecular oxygen to produce superoxide, which may undergo subsequent conversion to hydrogen peroxide under superoxide dismutase (SOD) mediation. The hydrogen peroxide may then undergo oxidation at the working electrode to provide a signal that may be correlated to the amount of ketones that were initially present. The SOD may be covalently bonded to a polymer in the ketones-responsive active area, according to various embodiments. Like the enzyme system shown in FIG. 2A, the β-hydroxybutyrate dehydrogenase and the NADH oxidase may be covalently bonded to a polymer in the ketones-responsive active area, and the NAD⁺/NADH may or may not be covalently bonded to a polymer in the ketones-responsive active area. If the NAD⁺ is not covalently bonded, it may be physically retained within the ketones-responsive active area, such as with a membrane polymer overcoating the ketones-responsive active area.

As shown in FIG. 2C, another enzymatic detection chemistry for ketones may utilize β-hydroxybutyrate dehydrogenase (HBDH) to convert β-hydroxybutyrate and NAD⁺ into acetoacetate and NADH, respectively. The electron transfer cycle in this case is completed by oxidation of NADH by 1,10-phenanthroline-5,6-dione to reform NAD⁺, wherein the 1,10-phenanthroline-5,6-dione subsequently transfers electrons to the working electrode. The 1,10-phenanthroline-5,6-dione may or may not be covalently bonded to a polymer within the ketones-responsive active area. Like the enzyme system shown in FIG. 2A, the β-hydroxybutyrate dehydrogenase may be covalently bonded to a polymer in the ketones-responsive active area, and the NAD⁺/NADH may or may not be covalently bonded to the polymer. Inclusion of an albumin in the ketones-responsive active area may provide a surprising improvement in the response stability. A suitable membrane polymer may promote retention of the NAD⁺ within the ketones-responsive active area.

Concurrent detection of an inhibitor of diaphorase and ketones may be particularly desirable due to the prevalence of diabetic individuals to experience ketoacidosis. As such, analyte sensors capable of analyzing for both a diaphorase inhibitor and ketones may facilitate treatment decisions and potentially improve therapeutic outcomes for such individuals. In addition to providing health benefits for diabetic individuals, analyte sensors featuring detection capabilities for both an inhibitor of diaphorase and ketones may be beneficial for other individuals who wish to monitor their ketones levels, such as individuals practicing a ketogenic diet. Ketogenic diets may be beneficial for promoting weight loss as well as helping epileptic individuals manage their condition. Coumarin-based drugs may sometimes be used by such individuals in response to cardiac health concerns.

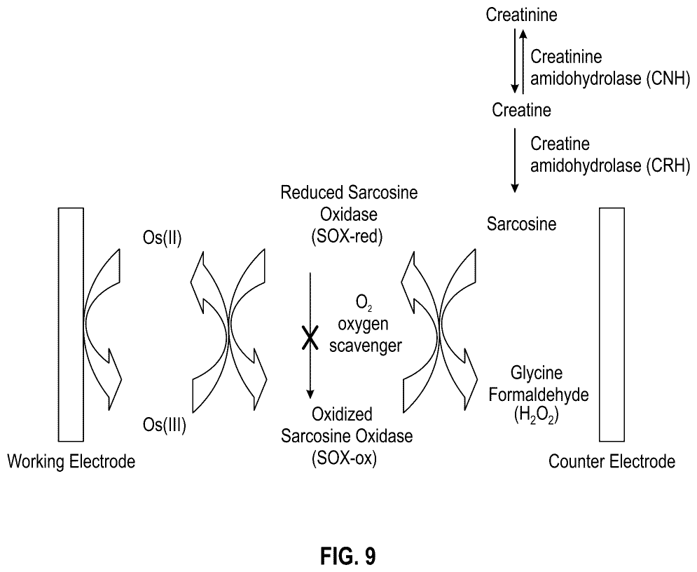

In some embodiments, the analyte sensors may further comprise a creatinine-responsive active area comprising an enzyme system that operates in concert to facilitate detection of creatinine. A suitable enzyme system that may be used for detecting creatinine in the analyte sensors disclosed herein is shown in FIG. 9 and described in further detail below. Additional details concerning enzyme systems responsive to creatinine may be found in commonly owned U.S. patent application Ser. No. 16/582,583 entitled “Analyte Sensors and Sensing Methods for Detecting Creatinine,” filed on Sep. 25, 2019, and published as U.S. Patent Application Publication 2020/0241015, the entirety of which is incorporated herein by reference.

As shown in FIG. 9, creatinine may react reversibly and hydrolytically in the presence of creatinine amidohydrolase (CNH) to form creatine. Creatine, in turn, may undergo catalytic hydrolysis in the presence of creatine amidohydrolase (CRH) to form sarcosine. Neither of these reactions produces a flow of electrons (e.g., oxidation or reduction) to provide a basis for electrochemical detection of the creatinine.

Referring still to FIG. 9, the sarcosine produced via hydrolysis of creatine may undergo oxidation in the presence of the oxidized form of sarcosine oxidase (SOX-ox) to form glycine and formaldehyde, thereby generating the reduced form of sarcosine oxidase (SOX-red) in the process. Hydrogen peroxide also may be generated in the presence of oxygen. The reduced form of sarcosine oxidase, in turn, may then undergo re-oxidation in the presence of the oxidized form of an electron transfer agent (e.g., an Os(III) complex), thereby producing the corresponding reduced form of the electron transfer agent (e.g., an Os(II) complex) and delivering a flow of electrons to the working electrode.

Oxygen may interfere with the concerted sequence of reactions used to detect creatinine in accordance with the disclosure above. Specifically, the reduced form of sarcosine oxidase may undergo a reaction with oxygen to reform the corresponding oxidized form of this enzyme but without exchanging electrons with the electron transfer agent. Although the enzymes all remain active when the reaction with oxygen occurs, no electrons flow to the working electrode. Without being bound by theory or mechanism, the competing reaction with oxygen is believed to result from kinetic effects. That is, oxidation of the reduced form of sarcosine oxidase with oxygen is believed to occur faster than does oxidation promoted by the electron transfer agent. Hydrogen peroxide is also formed in the presence of the oxygen.

The desired reaction pathway for facilitating detection of creatinine, shown in FIG. 9, may be encouraged by including an oxygen scavenger in proximity to the enzyme system. Various oxygen scavengers and dispositions thereof may be suitable, including oxidase enzymes such as glucose oxidase. Small molecule oxygen scavengers may also be suitable, but they may be fully consumed before the sensor lifetime is otherwise fully exhausted. Enzymes, in contrast, may undergo reversible oxidation and reduction, thereby affording a longer sensor lifetime. By discouraging oxidation of the reduced form of sarcosine oxidase with oxygen, the slower electron exchange reaction with the electron transfer agent may occur, thereby allowing production of a current at the working electrode. The magnitude of the current produced is proportional to the amount of creatinine that was initially reacted.

The oxygen scavenger used for encouraging the desired reaction pathway in FIG. 9 may be an oxidase enzyme in any embodiment of the present disclosure. Any oxidase enzyme may be used to promote oxygen scavenging in proximity to the enzyme system, provided that a suitable substrate for the enzyme is also present, thereby providing a reagent for reacting with the oxygen in the presence of the oxidase enzyme. Oxidase enzymes that may be suitable for oxygen scavenging in the present disclosure include, but are not limited to, glucose oxidase, lactate oxidase, xanthine oxidase, and the like. Glucose oxidase may be a particularly desirable oxidase enzyme to promote oxygen scavenging due to the ready availability of glucose in various bodily fluids. Reaction 1 below shows the enzymatic reaction promoted by glucose oxidase to afford oxygen clearing.

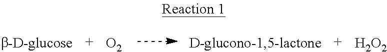

The concentration of available lactate in vivo is lower than that of glucose, but still sufficient to promote oxygen scavenging.

Oxidase enzymes, such as glucose oxidase, may be positioned in any location suitable to promote oxygen scavenging in the analyte sensors disclosed herein. Glucose oxidase, for example, may be positioned upon the sensor tail such that the glucose oxidase is functional and/or non-functional for promoting glucose detection. When non-functional for promoting glucose detection, the glucose oxidase may be positioned upon the sensor tail such that electrons produced during glucose oxidation are precluded from reaching the working electrode, such as through electrically isolating the glucose oxidase from the working electrode.

Concurrent detection of an inhibitor of diaphorase and creatinine may be particularly desirable due to the prevalence of diabetic individuals to experience diabetic neuropathy. By way of example, diabetic neuropathy may result from high blood glucose levels and lead to eventual kidney failure. Diabetic neuropathy is the leading cause of kidney failure in the United States and is experienced by a significant number of diabetic individuals within the first 10-20 years of their disease. Creatinine levels may be an analyte of particular interest for monitoring an individual's susceptibility to kidney failure, particularly due to diabetic neuropathy. As such, analyte sensors capable of analyzing for both a diaphorase inhibitor and creatinine may facilitate treatment decisions and potentially improve therapeutic outcomes for such individuals. Coumarin-based drugs may sometimes be used by individuals also having potential kidney failure concerns.

In some embodiments, the analyte sensors may further comprise a lactate-responsive active area comprising a lactate-responsive enzyme disposed upon the sensor tail. Suitable lactate-responsive enzymes may include, for example, lactate oxidase. Lactate oxidase or other lactate-responsive enzymes may be covalently bonded to a polymer comprising the lactate-responsive active area and exchange electrons with an electron transfer agent (e.g., an osmium (Os) complex or similar transition metal complex), which may also be covalently bonded to the polymer. Suitable electron transfer agents are described in further detail below. An albumin, such as human serum albumin, may be present in the lactate-responsive active area to stabilize the sensor response, as described in further detail in commonly owned U.S. Patent Application Publication 2019/0320947, which is incorporated herein by reference in its entirety. Lactate levels may vary in response to numerous environmental or physiological factors including, for example, eating, stress, exercise, sepsis or septic shock, infection, hypoxia, presence of cancerous tissue, or the like.

In some embodiments, the analyte sensors may further comprise an active area responsive to pH. Suitable analyte sensors configured for determining pH are described in commonly owned U.S. Patent Application Publication 2020/0060592, which is incorporated herein by reference in its entirety. Such analyte sensors may comprise a sensor tail comprising a first working electrode and a second working electrode, wherein a first active area located upon the first working electrode comprises a substance having pH-dependent oxidation-reduction chemistry, and a second active area located upon the second working electrode comprises a substance having oxidation-reduction chemistry that is substantially invariant with pH. By obtaining a difference between the first signal and the second signal, the difference may be correlated to the pH of a fluid to which the analyte sensor is exposed.

Accordingly, some embodiments of the analyte sensors disclosed herein may comprise a sensor tail comprising at least a first working electrode, and a first active area comprising an enzyme system responsive to an inhibitor of diaphorase and a second active area that is responsive to another analyte, such as a glucose-responsive active area, a lactate-responsive active area, a ketones-responsive active area, a creatinine-responsive active area, or a pH-responsive active area. The first active area responsive to the inhibitor of diaphorase and the other active area may be disposed upon the surface of the first working electrode and spaced apart from one another. Each active area may have an oxidation-reduction potential, wherein the oxidation-reduction potential of the first active area responsive to the inhibitor of diaphorase is sufficiently separated from the oxidation-reduction potential of the second active area to allow independent production of a signal from one of the active areas. By way of non-limiting example, the oxidation-reduction potentials may differ by at least about 100 mV, or by at least about 150 mV, or by at least about 200 mV. The upper limit of the separation between the oxidation-reduction potentials is dictated by the working electrochemical window in vivo. By having the oxidation-reduction potentials of the two active areas sufficiently separated in magnitude from one another, an electrochemical reaction may take place within one of the two active areas (i.e., within the first active area or the second active area) without substantially inducing an electrochemical reaction within the other active area. Thus, a signal from one of the first active area or the second active area may be independently produced at or above its corresponding oxidation-reduction potential (the lower oxidation-reduction potential) but below the oxidation-reduction potential of the other active area. A difference signal may allow the signal contribution from each analyte to be resolved.

Some or other embodiments of analyte sensors disclosed herein may feature the active area responsive to the inhibitor of diaphorase and the active area responsive to another analyte being located upon the surface of different working electrodes. Such analyte sensors may comprise a sensor tail comprising at least a first working electrode and a second working electrode, an active area responsive to the inhibitor of diaphorase disposed upon a surface of the first working electrode, and a second active area responsive to a different analyte disposed upon a surface of the second working electrode. A membrane may overcoat at least one of the first active area and the second active area. The membrane may be a mass transport limiting membrane and may comprise a multi-component membrane where the membrane overcoats at least one of the active areas. The multi-component membrane may comprise a bilayer of two different membrane polymers or an admixture of two different membrane polymers, wherein one of the membrane polymers overcoats the other active area.

An electron transfer agent may be present in any of the active areas disclosed herein, especially an active area responsive to an inhibitor of diaphorase and, if present, the active area responsive to another analyte. Suitable electron transfer agents may facilitate conveyance of electrons to the adjacent working electrode after one or more analytes undergoes an enzymatic oxidation-reduction reaction within the corresponding active area, thereby generating electron flow that is indicative of the presence of that particular analyte. The amount of current generated is proportional to the quantity of analyte that is present. Depending on the sensor configuration used, the electron transfer agents in the active area responsive to the inhibitor of diaphorase and the active area responsive to another analyte may be the same or different. For example, when two different active areas are disposed upon the same working electrode, the electron transfer agent within each active area may be different (e.g., chemically different such that the electron transfer agents exhibit different oxidation-reduction potentials). When multiple working electrodes are present, the electron transfer agent within each active area may be the same or different, since each working electrode may be interrogated separately.

Suitable electron transfer agents may include electroreducible and electrooxidizable ions, complexes or molecules (e.g., quinones) having oxidation-reduction potentials that are a few hundred millivolts above or below the oxidation-reduction potential of the standard calomel electrode (SCE). According to some embodiments, suitable electron transfer agents may include low-potential osmium complexes, such as those described in U.S. Pat. Nos. 6,134,461 and 6,605,200, which are incorporated herein by reference in their entirety. Additional examples of suitable electron transfer agents include those described in U.S. Pat. Nos. 6,736,957, 7,501,053 and 7,754,093, the disclosures of each of which are incorporated herein by reference in their entirety. Other suitable electron transfer agents may comprise metal compounds or complexes of ruthenium, osmium, iron (e.g., polyvinylferrocene or hexacyanoferrate), or cobalt, including metallocene compounds thereof, for example. Suitable ligands for the metal complexes may also include, for example, bidentate or higher denticity ligands such as, for example, bipyridine, biimidazole, phenanthroline, or pyridyl(imidazole). Other suitable bidentate ligands may include, for example, amino acids, oxalic acid, acetylacetone, diaminoalkanes, or o-diaminoarenes. Any combination of monodentate, bidentate, tridentate, tetradentate, or higher denticity ligands may be present in a metal complex to achieve a full coordination sphere.

Active areas suitable for detecting any of the analytes disclosed herein may comprise a polymer to which the electron transfer agents are covalently bound. Any of the electron transfer agents disclosed herein may comprise suitable functionality to promote covalent bonding to the polymer within the active areas. Suitable examples of polymer-bound electron transfer agents may include those described in U.S. Pat. Nos. 8,444,834, 8,268,143 and 6,605,201, the disclosures of which are incorporated herein by reference in their entirety. Suitable polymers for inclusion in the active areas may include, but are not limited to, polyvinylpyridines (e.g., poly(4-vinylpyridine)), polyvinylimidazoles (e.g., poly(1-vinylimidazole)), or any copolymer thereof. Illustrative copolymers that may be suitable for inclusion in the active areas include those containing monomer units such as styrene, acrylamide, methacrylamide, or acrylonitrile, for example. The polymer within each active area may be the same or different.

In particular embodiments of the present disclosure, the mass transport limiting membrane overcoating at least one of the active areas may comprise a crosslinked polyvinylpyridine homopolymer or copolymer. The composition of the mass transport limiting membrane may be the same or different where the mass transport limiting membrane overcoats each active area. When membrane composition is different, the membrane may comprise a bilayer membrane or a homogeneous admixture of two different membrane polymers, one of which may be a crosslinked polyvinylpyridine homopolymer or copolymer. Suitable techniques for depositing a mass transport limiting membrane upon the active area(s) may include, for example, spray coating, painting, inkjet printing, stenciling, roller coating, dip coating, the like, and any combination thereof.

Covalent bonding of the electron transfer agent to a polymer comprising an active area may take place by polymerizing a monomer unit bearing a covalently bonded electron transfer agent, or the electron transfer agent may be reacted with the polymer separately after the polymer has already been synthesized. A bifunctional spacer may covalently bond the electron transfer agent to the polymer within the active area, with a first functional group being reactive with the polymer (e.g., a functional group capable of quaternizing a pyridine nitrogen atom or an imidazole nitrogen atom) and a second functional group being reactive with the electron transfer agent (e.g., a functional group that is reactive with a ligand coordinating a metal ion).

Similarly, one or more of the enzymes within the active areas may be covalently bonded to a polymer comprising an active area. When an enzyme system comprising multiple enzymes is present in a given active area, all of the multiple enzymes may be covalently bonded to the polymer in some embodiments, and in other embodiments, only a portion of the multiple enzymes may be covalently bonded to the polymer. For example, one or more enzymes comprising an enzyme system may be covalently bonded to the polymer and at least one enzyme may be non-covalently associated with the polymer, such that the non-covalently bonded enzyme is physically entrained within the polymer. Covalent bonding of the enzyme(s) to the polymer in a given active area may take place via a crosslinker introduced with a suitable crosslinking agent. Suitable crosslinking agents for reaction with free amino groups in the enzyme (e.g., with the free side chain amine in lysine) may include crosslinking agents such as, for example, polyethylene glycol diglycidyl ether (PEGDGE) or other polyepoxides, cyanuric chloride, N-hydroxysuccinimide, imidoesters, epichlorohydrin, or derivatized variants thereof. Suitable crosslinking agents for reaction with free carboxylic acid groups in the enzyme may include, for example, carbodiimides. The crosslinking of the enzyme to the polymer is generally intermolecular, but can be intramolecular in some embodiments. In particular embodiments, all of the enzymes within a given active area may be covalently bonded to a polymer.

The electron transfer agent and/or the enzyme(s) may be associated with the polymer in an active area through means other than covalent bonding as well. In some embodiments, the electron transfer agent and/or the enzyme(s) may be ionically or coordinatively associated with the polymer. For example, a charged polymer may be ionically associated with an oppositely charged electron transfer agent or enzyme(s). In still other embodiments, the electron transfer agent and/or the enzyme(s) may be physically entrained within the polymer without being bonded thereto. Physically entrained electron transfer agents and/or enzyme(s) may still suitably interact with a fluid to promote analyte detection without being substantially leached from the active areas.

The polymer within the active area(s) may be chosen such that outward diffusion of NAD⁺ or another cofactor not covalently bound to the polymer is limited. Limited outward diffusion of the cofactor may promote a reasonable sensor lifetime (days to weeks) while still allowing sufficient inward analyte diffusion to promote detection.

The active area(s) in the analyte sensors disclosed herein may comprise one or more discrete spots (e.g., one to about ten spots, or even more discrete spots), which may range in size from about 0.01 mm² to about 1 mm², although larger or smaller individual spots within the active areas are also contemplated herein. Active areas defined as continuous bands around a cylindrical electrode are also possible in the disclosure herein. When an active area responsive to an inhibitor of diaphorase and an active area responsive to a different analyte are present, the number and/or size of individual spots may be the same or different.

It is also to be appreciated that the sensitivity (output current) of the analyte sensors toward each analyte may be varied by changing the coverage (area or size) of the active areas, the areal ratio of the active areas with respect to one another, the identity, thickness and/or composition of a mass transport limiting membrane overcoating the active areas. Variation of these parameters may be conducted readily by one having ordinary skill in the art once granted the benefit of the disclosure herein.

In more specific embodiments, analyte sensors of the present disclosure may comprise a sensor tail that is configured for insertion into a tissue. Suitable tissues are not considered to be particularly limited and are addressed in more detail above. Similarly, considerations for deploying a sensor tail at a particular position within a given tissue, such as a dermal layer of the skin, are addressed above.

Detection methods for assaying an inhibitor of diaphorase may comprise: exposing an analyte sensor to a fluid comprising a substrate of a NAD-dependent dehydrogenase and an inhibitor of diaphorase; wherein the analyte sensor comprises a sensor tail comprising at least a first working electrode, a first active area disposed upon a surface of the first working electrode, in which the first active area comprises an electron transfer agent, an enzyme system comprising NAD⁺, NADH or any combination thereof; a NAD-dependent dehydrogenase; and diaphorase; and an analyte-permeable membrane overcoating at least the first active area; wherein transfer of electrons from the first active area to the first working electrode is rate-limiting with respect to the diaphorase, such that the first active area is responsive to the inhibitor; applying a potential to the first working electrode; obtaining a first signal at or above an oxidation-reduction potential of the first active area, the first signal being proportional to a concentration of the inhibitor in the fluid; and correlating the first signal to the concentration of the inhibitor in the fluid. Optionally, the analyte-permeable membrane overcoating the first active area may be omitted if the NAD or NADH can be adequately retained within the first active area, such as through physical entrainment or covalent bonding to a polymer comprising the first active area. Any inhibitor of diaphorase may be assayed with the analyte sensors disclosed herein, including those specified above. The transfer of electrons to the first working electrode may be made rate-limiting with respect to diaphorase in any suitable manner discussed above.

In particular examples, the NAD-dependent dehydrogenase may be NAD-dependent glucose dehydrogenase and the substrate is glucose. Since glucose is readily prevalent in biological fluids, this substrate/dehydrogenase combination may be particularly advantageous for providing a supply of electrons for facilitating detection of the inhibitor of diaphorase.

In some embodiments, the first signal may be correlated to a corresponding concentration of the inhibitor of diaphorase by consulting a lookup table or calibration curve. A lookup table for a particular inhibitor may be populated by assaying multiple samples having known inhibitor concentrations and recording the sensor response at each concentration. Similarly, a calibration curve for the inhibitor may be determined by plotting the analyte sensor response as a function of the inhibitor concentration and determining a suitable calibration function over the calibration range (e.g., by regression, particularly linear regression).

A processor may determine which sensor response value in a lookup table is closest to that measured for a sample having an unknown analyte concentration and then report the analyte concentration accordingly. In some or other embodiments, if the sensor response value for a sample having an unknown analyte concentration is between the recorded values in the lookup table, the processor may interpolate between two lookup table values to estimate the analyte concentration. Interpolation may assume a linear concentration variation between the two values reported in the lookup table. Interpolation may be employed when the sensor response differs a sufficient amount from a given value in the lookup table, such as a variation of about 10% or greater.

Likewise, according to some or other various embodiments, a processor may input the sensor response value for a sample having an unknown analyte concentration into a corresponding calibration function. The processor may then report the analyte concentration accordingly.

The sensor tail may further comprise a second working electrode having an active area responsive to an analyte differing from the inhibitor disposed thereon, such as a glucose-responsive active area. As such, the methods may further comprise: obtaining a second signal at or above an oxidation-reduction potential of the glucose-responsive active area that is proportional to a concentration of glucose in the fluid, and correlating the second signal to the concentration of glucose in the fluid. Other analytes may be analyzed similarly by using an appropriate active area and applied potential.

According to more specific embodiments, the first signal and the second signal may be measured at different times. Thus, in such embodiments, a potential may be alternately applied to the first working electrode and the second working electrode. In other specific embodiments, the first signal and the second signal may be measured simultaneously via a first channel and a second channel, in which case a potential may be applied to both working electrodes at the same time. In either case, the signal associated with each active area may then be correlated to the concentration of the inhibitor of diaphorase and another analyte, such as glucose or similar analyte, using a lookup table or a calibration function in a similar manner to that discussed above.

Embodiments disclosed herein include:

A. Analyte sensors responsive to an inhibitor of diaphorase. The analyte sensors comprise: a sensor tail comprising at least a first working electrode; and a first active area disposed upon a surface of the first working electrode, the first active area comprising an electron transfer agent and an enzyme system comprising: nicotinamide adenine dinucleotide (NAD), reduced NAD, or any combination thereof, a NAD-dependent dehydrogenase, and diaphorase; wherein transfer of electrons from the first active area to the first working electrode is rate-limiting with respect to the diaphorase, such that the first active area is responsive to an inhibitor of diaphorase.

B. Methods for assaying an inhibitor of diaphorase. The methods comprise: exposing an analyte sensor to a fluid comprising a substrate of a nicotinamide adenine dinucleotide (NAD)-dependent dehydrogenase and an inhibitor of diaphorase; wherein the analyte sensor comprises a sensor tail comprising at least a first working electrode, and a first active area disposed upon a surface of the first working electrode, the first active area comprising an electron transfer agent and an enzyme system comprising NAD, reduced NAD, or any combination thereof; the NAD-dependent dehydrogenase; wherein transfer of electrons from the first active area to the first working electrode is rate-limiting with respect to the diaphorase, such that the first active area is responsive to the inhibitor; applying a potential to the first working electrode; obtaining a first signal at or above an oxidation-reduction potential of the first active area, the first signal being proportional to a concentration of the inhibitor in the fluid; and correlating the first signal to the concentration of the inhibitor in the fluid.

Embodiment A may have one or more of the following additional elements in any combination:

Element 1: wherein the NAD-dependent dehydrogenase is NAD-dependent glucose dehydrogenase.

Element 2: wherein the first active area comprises the diaphorase in a rate-limiting amount with respect to transferring electrons to the first working electrode, the diaphorase is modified to become rate-limiting with respect to transferring electrons to the first working electrode, or any combination thereof. Element 3: wherein the inhibitor of diaphorase comprises at least one compound selected from the group consisting of warfarin, dicoumarol, N-methylmaleimide, diphenyleneiodonium, 5,6-dimethylxanthenone-4-acetic acid, flavone-8-acetic acid, dimethylbenzylalkammonium chloride, 7,8-dihydroxyflavone, chrysin, and any combination thereof.

Element 4: wherein the analyte sensor further comprises an analyte-permeable membrane overcoating at least the first active area; wherein the analyte-permeable membrane is permeable to the inhibitor.

Element 5: wherein the analyte sensor further comprises a second active area that is responsive to an analyte differing from the inhibitor.

Element 6: wherein the second active area is a glucose-responsive active area comprising a glucose-responsive enzyme disposed upon the sensor tail.

Element 6A: wherein an analyte permeable membrane permeable to glucose overcoats the second active area.

Element 7: wherein the analyte sensor further comprises a second working electrode, the second active area being disposed upon a surface of the second working electrode; and an analyte-permeable membrane overcoating the second active area.

Element 8: wherein the sensor tail is configured for insertion into a tissue.

Element 9: wherein at least the electron transfer agent, the diaphorase, and the NAD-dependent dehydrogenase are covalently bound to a polymer comprising the first active area.

Element 10: wherein the first active area further comprises an albumin.

By way of non-limiting example, exemplary combinations applicable to A include, but are not limited to: 1 and 2; 1 and 3; 1 and 4; 1 and 5; 1 and 6; 1, 6 and 6A; 1, 6, 6A and 7; 1 and 7; 1 and 8; 1 and 9; 1 and 10; 1, 2 and 3; 1, 2 and 4; 1, 2, 4 and 5; 1, 4, 5 and 7; 1, 2 and 9; 1, 2 and 10; 2 and 3; 2-4; 2 and 4; 2 and 5; 2, 4 and 5; 2 and 6; 2, 6 and 6A; 2, 6 and 7; 2, 6, 6A and 7; 2 and 7; 2 and 8; 2 and 9; 2 and 10; 2, 4, 5 and 7; 2, 3 and 4; 2, 3, 5 and 6; 2, 4, 5, 6 and 6A; 2, 4, 5 and 7; 3 and 4; 3 and 5; 3, 4 and 5; 3, 4, 5 and 6; 3, 4, 5, 6 and 6A; 3 and 7; 3 and 8; 3 and 9; 3 and 10; 4 and 5; 4, 5 and 6; 4, 5, 6 and 6A; 4 and 7; 4 and 8; 4 and 9; 4 and 10; 8 and 9; 8 and 10; and 9 and 10.

Embodiment B may have one or more of the following additional elements in any combination:

Element 11: wherein the NAD-dependent dehydrogenase is NAD-dependent glucose dehydrogenase and the substrate is glucose.

Element 12: wherein the first active area comprises the diaphorase in a rate-limiting amount with respect to transferring electrons to the first working electrode, the diaphorase is modified to become rate-limiting with respect to transferring electrons to the first working electrode, or any combination thereof.

Element 13: wherein the inhibitor comprises at least one compound selected from the group consisting of warfarin, dicoumarol, N-methylmaleimide, diphenyleneiodonium, 5,6-dimethylxanthenone-4-acetic acid, flavone-8-acetic acid, dimethylbenzylalkammonium chloride, 7,8-dihydroxyflavone, chrysin, and any combination thereof.

Element 14: wherein an analyte-permeable membrane overcoats at least the first active area, the analyte-permeable membrane being permeable to the inhibitor.

Element 15: wherein the sensor tail further comprises a second active area that is responsive to an analyte differing from the inhibitor.

Element 16: wherein the second active area is a glucose-responsive active area comprising a glucose-responsive enzyme disposed upon the sensor tail, the method further comprising: obtaining a second signal at or above an oxidation-reduction potential of the glucose-responsive active area, the second signal being proportional to a concentration of glucose in the fluid; and correlating the second signal to the concentration of glucose in the fluid.

Element 16A: wherein the substrate is glucose.

Element 16B: wherein an analyte permeable membrane permeable to glucose overcoats the second active area.

Element 17: wherein the second active area is disposed upon a surface of a second working electrode, a second potential being applied to the second working electrode to obtain a second signal at or above an oxidation-reduction potential of the second active area.

Element 18: wherein an analyte-permeable membrane overcoats the second active area.

Element 19: wherein the first signal and the second signal are obtained at different times.

Element 20: wherein the first signal and the second signal are obtained simultaneously via a first channel and a second channel.

Element 21: wherein at least the electron transfer agent, the diaphorase, and the NAD-dependent dehydrogenase are covalently bound to a polymer comprising the first active area.

Element 22: wherein the first active area further comprises an albumin.

Element 23: wherein the fluid is a biological fluid and the analyte sensor is exposed to the biological fluid in vivo.

By way of non-limiting example, exemplary combinations applicable to B include, but are not limited to: 11 and 12; 11 and 13; 11-13; 11 and 14; 11, 12 and 14; 11, 13 and 14; 11-14; 11 and 15; 11, 15 and 16; 11, 15, 16 and 16A; 11, 15, 16 and 16B; 11, 15 and 17; 11, 15, 17 and 18; 11, 15, 17, 18 and 19; 11, 15, 17, 18 and 20; 11 and 21; 11 and 22; 11 and 23; 11, 12 and 21; 11, 12, 13 and 21; 11, 12 and 23; 11, 12, 13 and 23; 12 and 13; 12 and 14; 12-14; 12 and 15; 12, 15 and 16; 12, 15, 16 and 16A; 12, 15, 16 and 16B; 12, 15 and 17; 12, 15, 17 and 18; 12, 13, 14 15 and 17; 12, 13, 14, 15, 17 and 18; 12, 15, 17, and 19; 12, 15, 17 and 20; 12 and 21; 12 and 22; 12 and 23; 13 and 14; 13 and 15; 13, 15 and 16; 13, 15, 16 and 16A; 13, 15, 16 and 16B; 13 and 17; 13, 17 and 18; 13, 17 and 19; 13, 17 and 20; 13 and 21; 13 and 22; 13 and 23; 14 and 15; 14-16; 14, 15, 16 and 16A; 14, 15, 16 and 16B; 14, 15 and 17; 14, 15, 17 and 18; 14, 15, 17 and 19; 14, 15, 17 and 20; 14 and 21; 14 and 22; 14 and 23; 15 and 16; 15, 16 and 16A; 15, 16 and 16B; 15 and 17; 15 and 18; 15, 17 and 18; 15, 17 and 19; 15, 17 and 20; 15 and 21; 15 and 22; 15 and 23; 21 and 22; 21 and 23; and 22 and 23.

To facilitate a better understanding of the embodiments described herein, the following examples of various representative embodiments are given. In no way should the following examples be read to limit, or to define, the scope of the invention.

### EXAMPLES

A poly(vinylpyridine)-bound transition metal complex having the structure shown in Formula 1 was prepared. Further details concerning this transition metal complex and electron transfer therewith is provided in commonly owned U.S. Pat. No. 6,605,200, which was incorporated by reference above. The subscripts for each monomer represent illustrative atomic ratios and are not indicative of any particular monomer ordering.

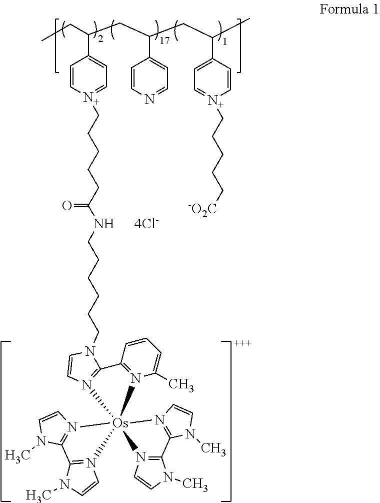

Example 1: Inhibition of Diaphorase by Dicoumarol. For this example, the spotting formulations shown in Table 1 below were coated onto separate carbon working electrodes. A micro-syringe was use to deposit 35 nL of each formulation as a single spot having an area of around 0.2 mm² upon separate carbon working electrodes. Following deposition, the working electrodes were cured overnight at 25° C.

TABLE 1
10 mM MES Buffer at pH = 5.5

| | Concentration (mg/mL) | | | |
|---|---|---|---|---|
| | Sensor | Sensor | Sensor | Sensor |
| Component | 1 | 2 | 3 | 4 |
| Diaphorase | 4 | 4 | 0.2 | 0.2 |
| Formula 1 | 8 | 0.4 | 8 | 0.4 |
| Polymer | | | | |
| PEGDGE400 | 4 | 4 | 4 | 4 |

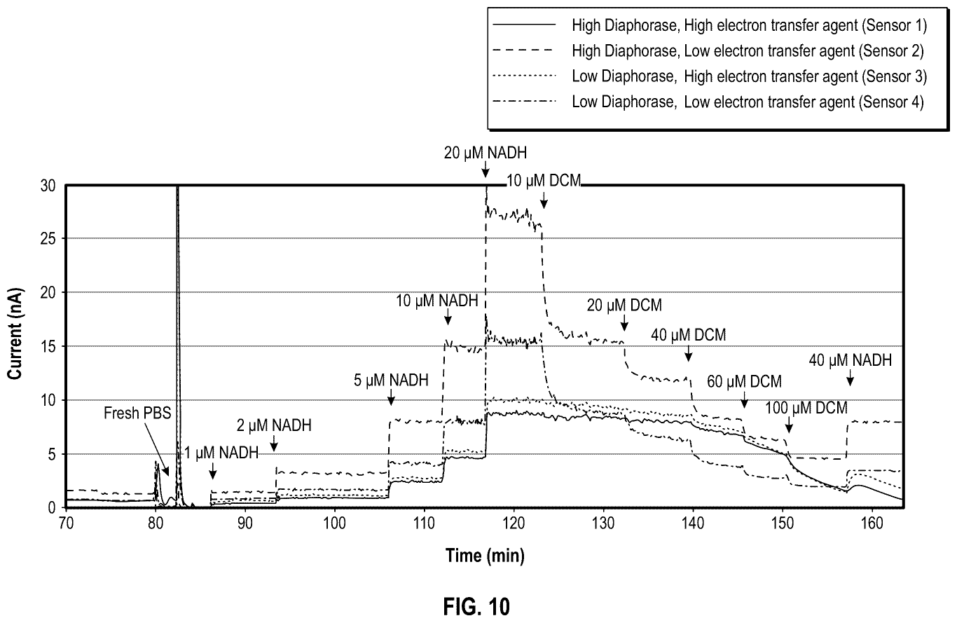

The electrodes were exposed to fresh phosphate buffered saline (PBS) solutions, and varying amounts of NADH and dicoumarol were then titrated into the buffered solutions. No glucose or GDH was added to “power” the sensors and complete the enzyme system specified above (FIG. 3B). NADH was titrated up to 30 μM. To the buffered solution containing 30 μM NADH was then titrated dicoumarol up to a concentration of 100 μM. After the dicoumarol concentration had been titrated to 100 μM, the NADH concentration was finally titrated to 40 μM. FIG. 10 is a graph showing the results of titrating NADH and dicoumarol (DCM) into PBS solutions exposed to the analyte sensors of Example 1 (Sensors 1-4). As shown, all the sensors were responsive to increasing NADH concentrations, but there was a sudden and sensitive decrease in signal as the dicoumarol concentration increased only for Sensors 2 and 4, each containing a low concentration of the electron transfer agent. A higher signal resulted for Sensor 2 as a result of its higher concentration of diaphorase. Given the goal of making the sensor response to be diaphorase limited, further optimization work centered around optimizing the sensor response at a low-concentration loading of the diaphorase.

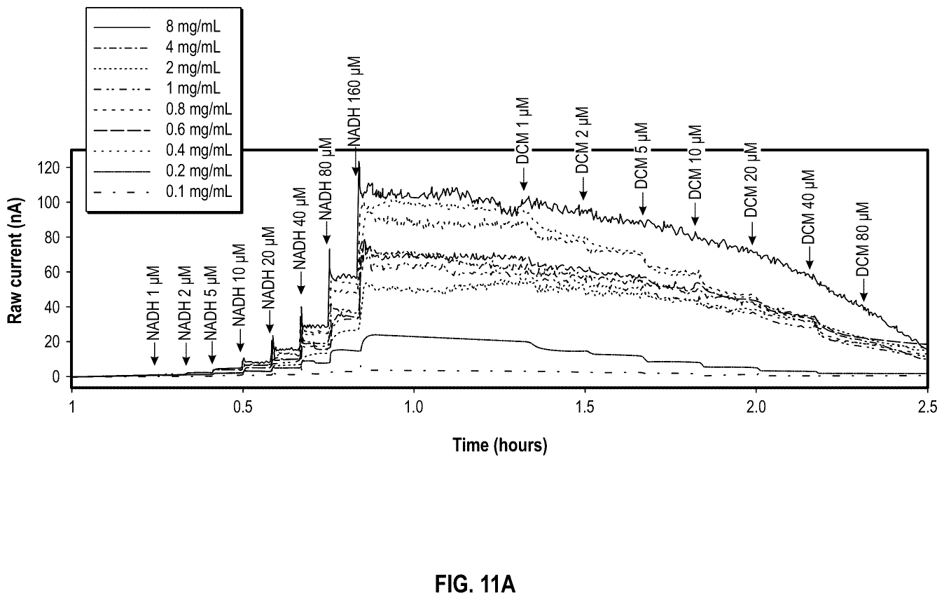

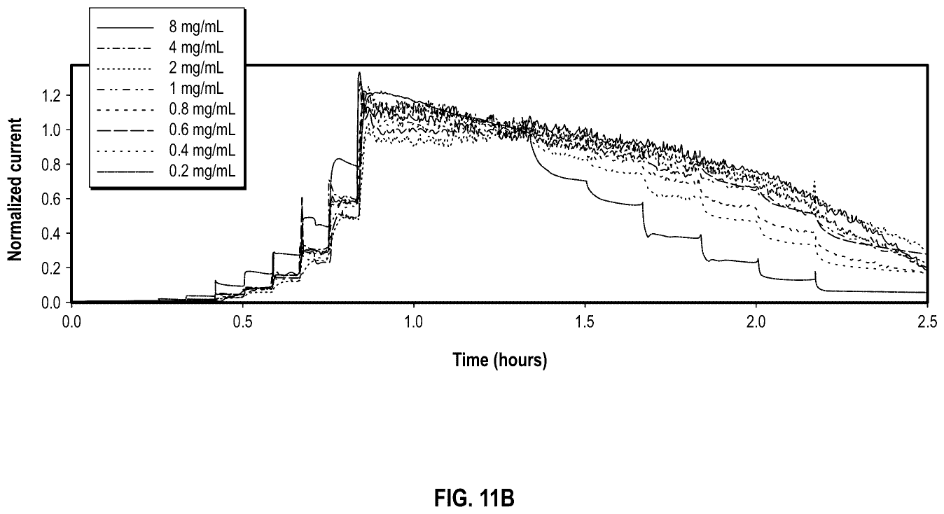

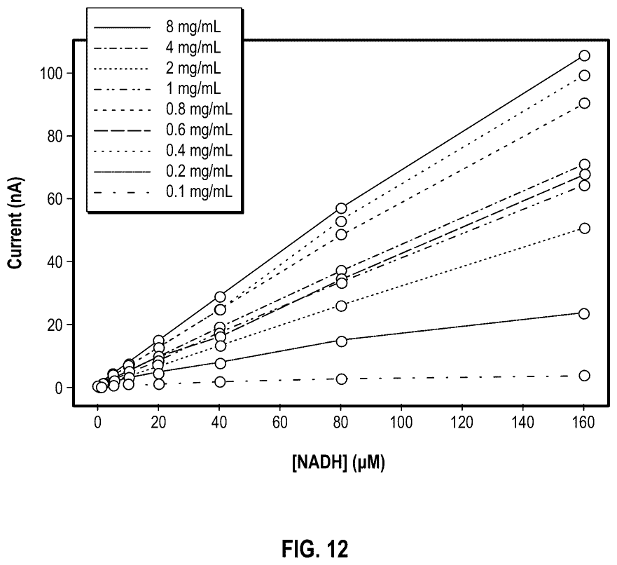

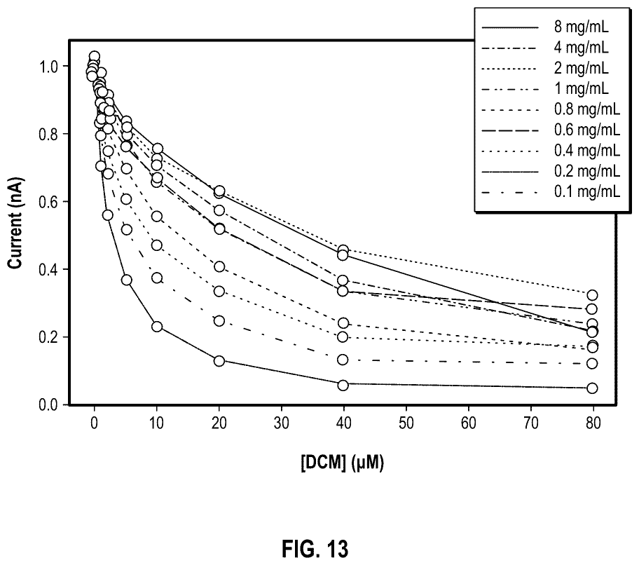

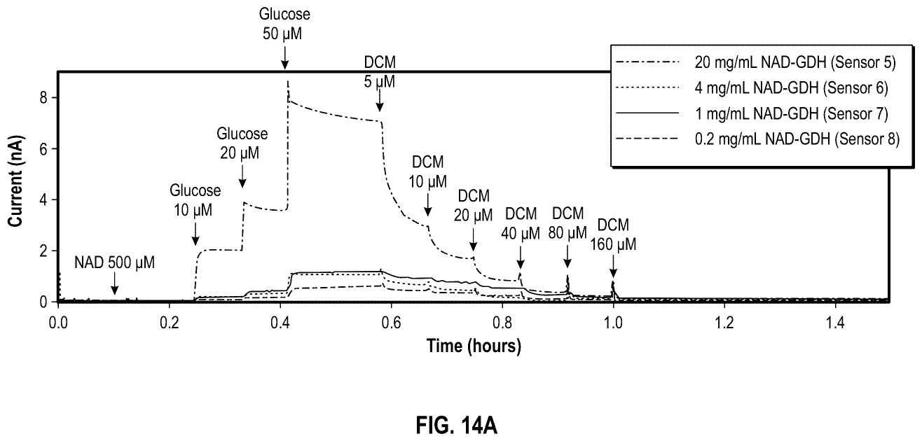

Next, several electrodes were fabricated as above using 10 mM MES buffer containing 0.2 mg/ml diaphorase, 4 mg/mL PEGDGE400, and varying amounts of the electron transfer agent (0.1, 0.2, 0.4, 0.6, 0.8, 1, 2, 4, and 8 mg/mL). NADH and dicoumarol were then titrated into PBS solutions exposed to the analyte sensors. In this case, NADH was titrated up to 160 μM, and dicoumarol was titrated up to 80 μM. FIGS. 11A and 11B are graphs showing the results of titrating NADH and dicoumarol (DCM) into PBS solutions exposed to electrodes having a variable amount of electron transfer agent in an active area thereon. FIG. 11A shows the raw current response, and FIG. 11B shows the normalized current response. FIG. 12 is a graph of sensor response as a function of NADH concentration for electrodes having a variable amount of electron transfer agent in an active area thereon, and FIG. 13 is a graph of normalized sensor response as a function of dicoumarol concentration for electrodes having a variable amount of electron transfer agent in an active area thereon. As shown, a concentration of 0.4 mg/ml of the electron transfer agent afforded an optimal combination of strong inhibition and good sensitivity for detection of the diaphorase inhibitor.

Example 2: Detection of Dicoumarol Using an Analyte Sensor Having an Active Area with Glucose Dehydrogenase and a Rate-Limited Electron Transfer to the Working Electrode. For this example, the spotting formulations shown in Table 2 below were coated onto separate carbon working electrodes. A micro-syringe was use to deposit 35 nL of each formulation as a single spot having an area of around 0.2 mm² upon separate carbon working electrodes. Following deposition, the working electrodes were cured overnight at 25° C.

TABLE 2
10 mM MES Buffer at pH = 5.5

| | Concentration (mg/mL) | | | |
|---|---|---|---|---|
| | Sensor | Sensor | Sensor | Sensor |
| Component | 5 | 6 | 7 | 8 |
| Diaphorase | 0.2 | 0.2 | 0.2 | 0.2 |
| GDH | 20 | 4 | 1 | 0.2 |
| NAD | 8 | 8 | 8 | 8 |
| Formula 1 | 0.4 | 0.4 | 0.4 | 0.4 |
| Polymer | | | | |
| HSA | 8 | 8 | 8 | 8 |
| PEGDGE400 | 4 | 4 | 4 | 4 |

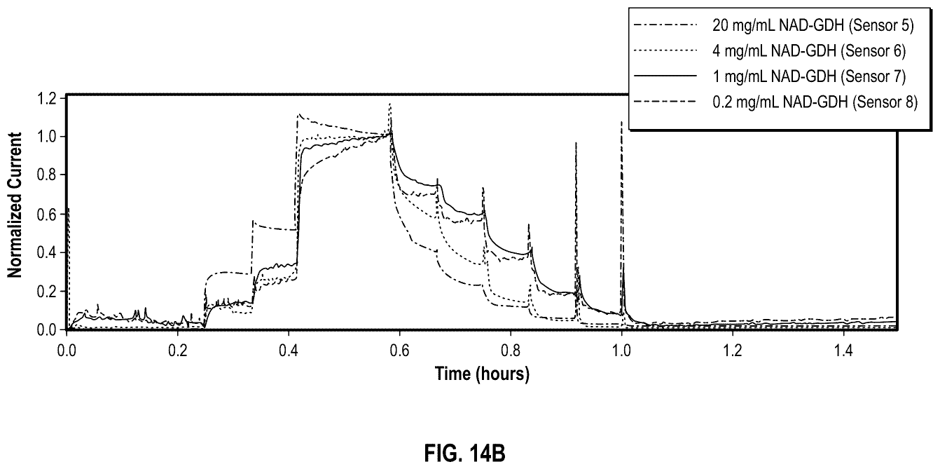

Glucose and dicoumarol were then titrated to PBS solutions in which the sensors were immersed. A 500 μM quantity of NAD was first added to the buffer solution, followed by three additions of glucose up to 50 μM. Thereafter, dicoumarol (DCM) was titrated up to 160 μM. FIGS. 14A and 14B are graphs showing the results of titrating glucose and dicoumarol (DCM) into PBS solutions exposed to electrodes having variable amounts of NAD-dependent glucose dehydrogenase in an active area thereon. FIG. 14A shows the raw current response, and FIG. 14B shows the normalized current response. As shown, increasing amounts of glucose dehydrogenase increased the signal response during glucose addition and during DCM addition. Overall, the DCM response decreased as increasing amounts of DCM were added.

Unless otherwise indicated, all numbers expressing quantities and the like in the present specification and associated claims are to be understood as being modified in all instances by the term “about.” Accordingly, unless indicated to the contrary, the numerical parameters set forth in the specification and attached claims are approximations that may vary depending upon the desired properties sought to be obtained by the embodiments of the present invention. At the very least, and not as an attempt to limit the application of the doctrine of equivalents to the scope of the claim, each numerical parameter should at least be construed in light of the number of reported significant digits and by applying ordinary rounding techniques.

One or more illustrative embodiments incorporating various features are presented herein. Not all features of a physical implementation are described or shown in this application for the sake of clarity. It is understood that in the development of a physical embodiment incorporating the embodiments of the present invention, numerous implementation-specific decisions must be made to achieve the developer's goals, such as compliance with system-related, business-related, government-related and other constraints, which vary by implementation and from time to time. While a developer's efforts might be time-consuming, such efforts would be, nevertheless, a routine undertaking for those of ordinary skill in the art and having benefit of this disclosure.

While various systems, tools and methods are described herein in terms of “comprising” various components or steps, the systems, tools and methods can also “consist essentially of” or “consist of” the various components and steps.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Therefore, the disclosed systems, tools and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems, tools and methods illustratively disclosed herein may suitably be practiced in the absence of any element that is not specifically disclosed herein and/or any optional element disclosed herein. While systems, tools and methods are described in terms of “comprising,” “containing,” or “including” various components or steps, the systems, tools and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

1.-11. (canceled)

12. A method of detecting a concentration of an inhibitor of diaphorase in a fluid, the method comprising:
exposing an analyte sensor to a fluid comprising a substrate of a nicotinamide adenine dinucleotide (NAD)-dependent dehydrogenase and an inhibitor of diaphorase;
wherein the analyte sensor comprises at least a first working electrode, and a first active area disposed upon a surface of the first working electrode, the first active area comprising an electron transfer agent and an enzyme system comprising:
a substrate of the NAD-dependent dehydrogenase;
NAD, reduced NAD, or any combination thereof;
the NAD-dependent dehydrogenase; and
diaphorase;
wherein the substrate of the NAD-dependent dehydrogenase provides a steady flow of electrons from the first active area to the first working electrode;
wherein transfer of electrons from the first active area to the first working electrode is rate-limiting with respect to the diaphorase, such that the first active area is responsive to the inhibitor of diaphorase;
applying a potential to the first working electrode;
obtaining a first signal at or above an oxidation-reduction potential of the first active area, the first signal being proportional to a concentration of the inhibitor of diaphorase in the fluid; and
correlating the first signal to the concentration of the inhibitor of diaphorase in the fluid.

13. The method of claim 12, wherein the NAD-dependent dehydrogenase is NAD-dependent glucose dehydrogenase and the substrate is glucose.

14. The method of claim 12, wherein the first active area comprises the diaphorase in a rate-limiting amount with respect to transferring electrons to the first working electrode, the diaphorase is modified to become rate-limiting with respect to transferring electrons to the first working electrode, or any combination thereof.

15. The method of claim 12, wherein the first active area is responsive to at least one inhibitor of diaphorase selected from the group consisting of warfarin, dicoumarol, N-methylmaleimide, diphenyleneiodonium, 5,6-dimethylxanthenone-4-acetic acid, flavone-8-acetic acid, dimethylbenzylalkammonium chloride, 7,8-dihydroxyflavone, chrysin, and any combination thereof.

16. The method of claim 12, wherein an analyte-permeable membrane overcoats at least the first active area, the analyte-permeable membrane being permeable to the inhibitor of diaphorase.

17. The method of claim 12, wherein the analyte sensor further comprises a second active area that is responsive to an analyte differing from the inhibitor of diaphorase.

18. The method of claim 17, wherein the second active area is a glucose-responsive active area comprising a glucose-responsive enzyme disposed upon the second working electrode, the method further comprising:
obtaining a second signal at or above an oxidation-reduction potential of the glucose-responsive active area, the second signal being proportional to a concentration of glucose in the fluid; and
correlating the second signal to the concentration of glucose in the fluid.

19. The method of claim 17, wherein the second active area is disposed upon a surface of a second working electrode, a second potential being applied to the second working electrode to obtain a second signal at or above an oxidation-reduction potential of the second active area.

20. The method of claim 19, wherein an analyte-permeable membrane overcoats the second active area.

21. The method of claim 19, wherein the first signal and the second signal are obtained at different times.

22. The method of claim 19, wherein the first signal and the second signal are obtained simultaneously via a first channel and a second channel.

23. The method of claim 12, wherein at least the electron transfer agent, the diaphorase, and the NAD-dependent dehydrogenase are covalently bound to a polymer comprising the first active area.

24. The method of claim 12, wherein the first active area further comprises an albumin.

25. The method of claim 12, wherein the fluid is a biological fluid and the analyte sensor is exposed to the biological fluid in vivo.

26. An analyte sensor comprising:
a first working electrode; and
a first active area suitable for detecting an inhibitor of diaphorase, the first active area disposed upon a surface of the first working electrode, the first active area comprising an electron transfer agent and an enzyme system comprising:
a substrate of a nicotinamide adenine dinucleotide (NAD) dehydrogenase;
NAD, reduced NAD, or any combination thereof;
an NAD-dependent dehydrogenase; and
diaphorase;
wherein the substrate of the NAD-dependent dehydrogenase provides a steady flow of electrons from the first active area to the first working electrode,
wherein transfer of electrons from the first active area to the first working electrode is rate-limiting with respect to the diaphorase, such that the first active area is responsive to the inhibitor of diaphorase, wherein the first working electrode receives an electrochemical signal that correlates with concentration of the inhibitor of diaphorase, and
wherein the diaphorase and the NAD, reduced NAD, or any combination thereof are present in a weight ratio in the range of 1:40 to 1:2.

27. The analyte sensor of claim 26, wherein the NAD-dependent dehydrogenase is NAD-dependent glucose dehydrogenase and the substrate is glucose.

28. The analyte sensor of claim 26, wherein the first active area comprises the diaphorase in a rate-limiting amount with respect to transferring electrons to the first working electrode, the diaphorase is modified to become rate-limiting with respect to transferring electrons to the first working electrode, or any combination thereof.

29. The analyte sensor of claim 26, wherein the first active area is responsive to at least one inhibitor of diaphorase selected from the group consisting of warfarin, dicoumarol, N-methylmaleimide, diphenyleneiodonium, 5,6-dimethylxanthenone-4-acetic acid, flavone-8-acetic acid, dimethylbenzylalkammonium chloride, 7,8-dihydroxyflavone, chrysin, and any combination thereof.

30. The analyte sensor of claim 26, further comprising:
an analyte-permeable membrane overcoating at least the first active area;
wherein the analyte-permeable membrane is permeable to the inhibitor of diaphorase.

31. The analyte sensor of claim 26, further comprising:
a second active area that is responsive to an analyte differing from the inhibitor of diaphorase.

32. The analyte sensor of claim 31, wherein the second active area is a glucose-responsive active area comprising a glucose-responsive enzyme disposed upon the substrate.

33. The analyte sensor of claim 31, further comprising:
a second working electrode, the second active area being disposed upon a surface of the second working electrode; and
an analyte-permeable membrane overcoating the second active area.

34. The analyte sensor of claim 26, wherein the substrate is configured for insertion into a tissue.

35. The analyte sensor of claim 26, wherein at least the electron transfer agent, the diaphorase, and the NAD-dependent dehydrogenase are covalently bound to a polymer comprising the first active area.

36. The analyte sensor of claim 26, wherein the first active area further comprises an albumin.
